(19) United States
(12) Patent Application Publication
(10) Pub. No.: US 2008/0003634 A1
(43) Pub. Date: Jan. 3, 2008

(54) Extended Primary Retinal Cell Culture and Stress Models, and Methods of Use

(75) Inventor: Ryo Kubota, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: ACUCELA, INC., Bothell, WA (US)

(21) Appl. No.: 11/758,480

(22) Filed: Jun. 5, 2007

Related U.S. Application Data
(63) Continuation of application No. 10/903,880, filed on Jul. 30, 2004, now abandoned.
(60) Provisional application No. 60/491,412, filed on Jul. 30, 2003.
(60) Provisional application No. 60/491,904, filed on Aug. 1, 2003.
(60) Provisional application No. 60/561,029, filed on Apr. 9, 2004.

(51) Int. Cl. C12N 5/06; C12Q 1/02

(52) U.S. Cl. 435/29; 435/347

(57) ABSTRACT

A cell culture system related to extended in vitro culture of mature retinal cells and methods for preparing the cell culture system are provided. Also provided is a retinal cell culture stress model related to extended in vitro culture of mature retinal cells in the presence of a stressor and methods for using the cell culture stress model. The invention provides a cell culture system comprising a long-term culture of mature retinal cells, without requiring addition of other types of non-retinal cells such as purified glia, or cells isolated from ciliary bodies within the eye, and the addition of a stressor such as light, A2E, cigarette smoke condensate, glutamate, or hydrostatic pressure. Methods for identifying bioactive agents that alter viability, neurodegeneration, or survival of retinal cells using the retinal cell culture stress system are also provided.

### CROSS-REFERENCE TO RELATED APPLICATIONS

This application is a continuation of U.S. patent application Ser. No. 10/903,880, filed Jul. 30, 2004 (now pending), which claims the benefit of U.S. Provisional Patent Application No. 60/491,412 filed Jul. 30, 2003; U.S. Provisional Patent Application No. 60/491,904, filed Aug. 1, 2003; and U.S. Provisional Patent Application No. 60/561,029, filed Apr. 9, 2004, all of which are incorporated herein by reference in their entireties.

### BACKGROUND OF THE INVENTION

1. Field of the Invention

The present invention relates generally to a cell culture system that provides extended in vitro culture of retinal cells and to a cell culture system comprising a neuronal cell stressor that provides a model for determining the effects of the stressor on the extended in vitro culture of retinal cells. The invention is particularly related to a cell culture stress model comprising retinal neuronal cells including photoreceptor, amacrine, bipolar, horizontal, and ganglion cells. The cell culture model is useful for identifying bioactive agents that can be used for treating neurodegenerative diseases, particularly retinal diseases and disorders.

2. Description of the Related Art

Neurodegenerative diseases, such as glaucoma, macular degeneration, and Alzheimer's disease, affect millions of patients throughout the world. Because the loss of quality of life associated with these diseases is considerable, drug research and development in this area is of great importance.

Macular degeneration is a disease that affects central vision. Macular degeneration affects between five and ten million patients in the United States, and it is the leading cause of blindness worldwide. Macular degeneration is a disease that causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease usually leads to more serious vision loss. The exact causes of age-related macular degeneration are still unknown. The dry form of ARMD may result from the aging and thinning of macular tissues and from deposition of pigment in the macula. In wet ARMD, new blood vessels grow beneath the retina and leak blood and fluid. This leakage causes the retinal cells to die, creating blind spots in central vision.

The only Food and Drug Administration (FDA)-approved protocol for treating ARMD is a photodynamic therapy that uses a special drug combined with laser photocoagulation. This treatment, however, can only be applied to half of patients newly diagnosed with wet form of ARMD. For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Declining vision noticed by the patient or by an ophthalmologist during a routine eye exam may be the first indicator of macular degeneration. The formation of exudates, or “drusen,” from blood vessels in and under the macular is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes.

Different forms of macular degeneration may also occur in younger patients. Non-age related etiology may be linked to heredity, diabetes, nutritional deficits, head injury, infection, or other factors.

Glaucoma is a broad term used to describe a group of diseases that causes visual field loss, often without any other prevailing symptoms. The lack of symptoms often leads to a delayed diagnosis of glaucoma until the terminal stages of the disease. Prevalence of glaucoma is estimated to be three million in the United States, with about 120,000 cases of blindness attributable to the condition. The disease is also prevalent in Japan, which has four million reported cases. In other parts of the world, treatment is less accessible than in the United States and Japan, thus glaucoma ranks as a leading cause of blindness worldwide. Even if subjects afflicted with glaucoma do not become blind, their vision is often severely impaired.

The loss of peripheral vision is caused by the death of ganglion cells in the retina. Ganglion cells are a specific type of projection neuron that connects the eye to the brain. Glaucoma is often accompanied by an increase in intraocular pressure. Current treatment includes use of drugs that lower the intraocular pressure; however, lowering the intraocular pressure is often insufficient to completely stop disease progression. Ganglion cells are believed to be susceptible to pressure and may suffer permanent degeneration prior to the lowering of intraocular pressure. An increasing number of cases of normal tension glaucoma has been observed in which ganglion cells degenerate without an observed increase in the intraocular pressure. Because current glaucoma drugs only treat intraocular pressure, a need exists to identify new therapeutic agents that will prevent or reverse the degeneration of ganglion cells. Recent reports suggest that glaucoma is a neurodegenerative disease, similar to Alzheimer's disease and Parkinson's disease in the brain, except that it specifically affects retinal neurons. The retinal neurons of the eye originate from diencephalon neurons of the brain. Though retinal neurons are often mistakenly thought not to be part of the brain, retinal cells are key components of vision, interpreting the signals from the light sensing cells.

Alzheimer's disease (AD) is the most common form of dementia among the elderly. Dementia is a brain disorder that seriously affects a person's ability to carry out daily activities. Alzheimer's is a disease that affects four million people in the United States alone. It is characterized by a loss of nerve cells in areas of the brain that are vital to memory and other mental functions. Some drugs can prevent AD symptoms for a finite period of time, but no drugs are available that treat the disease or completely stop the progressive decline in mental function. Recent research suggests that glial cells that support the neurons or nerve cells may have defects in AD sufferers, but the cause of AD remains unknown. Individuals with AD seem to have a higher incidence of glaucoma and macular degeneration, indicating that similar pathogenesis may underlie these neurodegenerative diseases of the eye and brain. (See Giasson et al., Free Radic. Biol. Med. 32:1264-75 (2002); Johnson et al., Proc. Natl. Acad. Sci. USA 99:11830-35 (2002); Dentchev et al., Mol. Vis. 9:184-90 (2003)).

Neuronal cell death underlies the pathology of these diseases. Unfortunately, very few compositions and methods that enhance neuronal cell survival, particularly photoreceptor cell survival, have been discovered. The lack of a good animal model has proved to be a major obstacle for developing new drugs to treat retinal diseases and disorders. For example, macula exist in primates (including humans) but not in rodents; therefore, relatively less expensive, well-developed rodent animal models are currently not available for testing drugs and biologicals that directly target the macula. Alternative methods to animal models for identifying and evaluating compositions and methods of treatment of retinal diseases are therefore needed in the art.

In vitro culture of neuronal cells in general, and of retinal neuronal cells in particular, has been problematic. For many years, fully mature neurons were thought to lack plasticity and the ability to repair and regenerate after injury. If mature central nervous system (CNS) neurons could be cultured in vitro over an extended period of time and also be stimulated to regenerate, transplantation and functional restoration of damaged or diseased CNS tissue might become feasible.

Groups of investigators have been studying in vitro growth of CNS-derived neurons. Some studies have involved use of transformed or immortalized neuronal cells, including cells derived from tumorigenic tissues. With respect to culturing retinal neuronal cells, in vitro retinal organ cultures, retinal explant cultures, and retinal explant/membrane culture techniques have been reported. In addition, investigators have reported analysis of retinal neuronal cell cultures that are derived from embryonic tissue or embryonic stem cells or from neonatal retinas. The inability to establish long-term culture of post-mitotic neuronal cells, however, has been a major roadblock within the field of neurobiology. A valuable contribution to the neurobiology arts would include the development of a cell culture model that includes stressors that affect cells in an in vitro cell culture similarly to how stressors affect cells in vivo.

### BRIEF SUMMARY OF THE INVENTION

Briefly stated, the present invention provides a mature retinal cell culture system, a retinal cell culture stress model comprising the mature retinal cell culture system, and provides methods for using the mature retinal cell culture system and stress model.

In one embodiment, the invention provides a cell culture system comprising a plurality of mature retinal cells and at least one cell stressor, wherein the cell stressor reduces viability of the mature retinal cells. In certain embodiments, the cell stressor is light, retinoid N-retinylidene-N-retinyl-ethanolamine (A2E) or an isoform thereof, cigarette smoke condensate, increased hydrostatic pressure, or glutamate. In a particular embodiment, the cell stressor is light, which may be blue light or white light. In one particular embodiment, the blue light has an intensity between about 250-8000 lux. In another particular embodiment, the white light has an intensity between about 250-8000 lux. In another embodiment, the light may be ultraviolet light. In another particular embodiment, the light is emitted from a fluorescent bulb, an incandescent bulb, or a light emitting diode. In other particular embodiments, the retinal cell stressor is increased atmospheric pressure. In one embodiment, the stressor is a chemical, and in a certain embodiment the chemical is A2E, which may include an isomer of A2E. In another embodiment, the chemical is glutamate or a glutamate agonist. In another embodiment, the cell culture system comprises at least two retinal cell stressors, which may be selected from light, A2E or an isoform thereof, cigarette smoke condensate, increased hydrostatic pressure, or glutamate. In a certain embodiment, the at least two cell stressors are light and A2E, and in another embodiment, the at least two cell stressors are light and cigarette smoke condensate.

In a certain embodiment, the cell culture system comprises a plurality of mature retinal cells and at least one cell stressor, wherein the plurality of mature retinal cells comprises at least one retinal neuronal cell, at least one retinal pigmented epithelial cell, and at least one Müller glial cell. In certain embodiments, the plurality of retinal cells comprises a plurality of retinal neuronal cells comprising at least one bipolar cell, at least one horizontal cell, at least one amacrine cell, at least one ganglion cell, and at least one photoreceptor cell. In another embodiment, the cell culture system comprises a plurality of mature retinal cells and at least one cell stressor, wherein the plurality of mature retinal cells comprises at least one retinal neuronal cell; wherein the retinal neuronal cell is a bipolar cell, horizontal cell, amacrine cell, ganglion cell, or photoreceptor cell. In another embodiment, the plurality of mature retinal cells comprises at least one cell selected from a retinal neuronal cell, a retinal pigmented epithelial cell, and a Müller glial cell, wherein the retinal neuronal cell is a bipolar cell, a horizontal cell, an amacrine cell, a ganglion cell, or a photoreceptor cell. In another embodiment, the cell culture system is substantially free of cells purified from a non-retinal tissue source.

In another embodiment, the invention provides a cell culture system comprising a plurality of mature retinal cells, wherein the cell culture system is substantially free of cells purified from a non-retinal tissue source, and wherein the plurality of mature retinal cells comprises at least one retinal neuronal cell, at least one retinal pigmented epithelial cell, and at least one Müller glial cell. In certain embodiments, the plurality of mature retinal cells comprises a plurality of retinal neuronal cells comprising at least one bipolar cell, at least one horizontal cell, at least one amacrine cell, at least one ganglion cell, and at least one photoreceptor cell. In one embodiment, the plurality of mature retinal cells comprises at least one cell selected from a retinal neuronal cell, a retinal pigmented epithelial cell, and a Müller glial cell. In a specific embodiment, the plurality of mature retinal cells comprises at least one retinal neuronal cell selected from a bipolar cell, a horizontal cell, an amacrine cell, a ganglion cell, or a photoreceptor cell. In particular embodiments, the plurality of mature retinal cells are viable for at least 2 weeks, at least 4 weeks, at least 8 weeks, at least 12 weeks, or at least 16 weeks. In another embodiment, the invention provides a method for producing the cell culture system comprising isolating mature retinal cells from a biological source and culturing the mature retinal cells under conditions that maintain viability of the mature retinal cells, wherein the biological source is retinal tissue from a bird or a mammal, which mammal is a human, pig, non-human primate, an ungulate, a dog, or a rodent.

In another embodiment, the invention provides a method for identifying a stressor of mature retinal cells comprising (a) contacting a candidate stressor and the cell culture system comprising a plurality of mature retinal cells, wherein the cell culture system is substantially free of cells purified form a non-retinal source, under conditions and for a time sufficient to permit interaction between the candidate stressor and a mature retinal cell; and (b) comparing viability of a mature retinal cell in the presence of the candidate stressor with viability of a mature retinal cell in the absence of the candidate stressor, and therefrom identifying a stressor of retinal cells. In one embodiment, viability is determined by comparing a level of survival of the mature retinal cell in the presence of the candidate stressor with a level of survival of the mature retinal cell in the absence of the candidate stressor, wherein decreased (not extended) survival in the presence of the candidate agent indicates that the stressor decreases viability of the retinal cells. In another embodiment, viability is determined by comparing neurodegeneration of the mature retinal cell in the presence of the candidate stressor with neurodegeneration of the mature retinal cell in the absence of the candidate stressor, wherein enhancement of neurodegeneration in the presence of the candidate stressor indicates that the stressor decreases viability of the retinal cell. In a certain embodiment, the step of comparing viability of the mature retinal cell comprises determining viability of at least one (a) retinal neuronal cell selected from a bipolar cell, a horizontal cell, an amacrine cell, a ganglion cell, and a photoreceptor cell; (b) one retinal pigmented epithelial cell; or (c) one Müller glial cell.

The present invention also provides a method for identifying a bioactive agent that alters viability of a mature retinal cell comprising: (a) contacting a candidate agent and (1) the cell culture system comprising a plurality of mature retinal cells, wherein the cell culture system is substantially free of cells purified form a non-retinal source, or (2) the cell culture system comprising a plurality of mature retinal cells and at least one cell stressor, wherein the cell stressor reduces viability of the mature retinal cells, under conditions and for a time sufficient to permit interaction between a mature retinal cell of the cell culture system and the candidate agent; and (b) comparing viability of a mature retinal cell in the presence of the candidate agent with viability of a mature neuronal cell in the absence of the candidate agent, therefrom identifying a bioactive agent that is capable of altering viability of a retinal cell. In one embodiment, viability is determined by comparing a level of survival of the mature retinal cell in the presence of the candidate agent with a level of survival of the mature retinal cell in the absence of the candidate agent, wherein increased survival in the presence of the candidate agent indicates that the agent increases viability of the retinal cell. In another embodiment, viability is determined by comparing neurodegeneration of the mature retinal cell in the presence of the candidate agent with neurodegeneration of the mature retinal cell in the absence of the candidate agent, wherein inhibition of neurodegeneration in the presence of the candidate agent indicates that the agent increases viability of the retinal cell. In a certain embodiment, the step of comparing viability of the mature retinal cell comprises determining viability of at least one (a) retinal neuronal cell selected from a bipolar cell, a horizontal cell, an amacrine cell, a ganglion cell, and a photoreceptor cell; (b) one retinal pigmented epithelial cell; or (c) one Müller glial cell.

The invention also provides a method for identifying a bioactive agent capable of treating a retinal disease comprising (a) contacting a candidate agent and (1) the cell culture system comprising a plurality of mature retinal cells, wherein the cell culture system is substantially free of cells purified form a non-retinal source, or (2) the cell culture system comprising a plurality of mature retinal cells and at least one cell stressor, wherein the cell stressor reduces viability of the mature retinal cells, under conditions and for a time sufficient to permit interaction between a mature retinal cell of the cell culture system and the candidate agent; and (b) comparing viability of a mature retinal cell in the presence of the candidate agent with viability of a mature neuronal cell in the absence of the candidate agent, therefrom identifying a bioactive agent that is capable of treating a retinal disease. In one embodiment, viability is determined by comparing a level of survival of the mature retinal cell in the presence of the candidate agent with a level of survival of the mature retinal cell in the absence of the candidate agent, wherein increased survival in the presence of the candidate agent indicates that the agent increases viability of the retinal cell. In another embodiment, viability is determined by comparing neurodegeneration of the mature retinal cell in the presence of the candidate agent with neurodegeneration of the mature retinal cell in the absence of the candidate agent, wherein inhibition of neurodegeneration in the presence of the candidate agent indicates that the agent increases viability of the retinal cell. In particular embodiments, the retinal disease is macular degeneration, glaucoma, diabetic retinopathy, retinal detachment, retinal blood vessel occlusion, retinitis pigmentosa, optic neuropathy, inflammatory retinal disease, or a retinal disorder associated with Alzheimer's disease, Parkinson's disease, or multiple sclerosis. In a certain embodiment, the step of comparing viability of the mature retinal cell comprises determining viability of at least one (a) retinal neuronal cell selected from a bipolar cell, a horizontal cell, an amacrine cell, a ganglion cell, and a photoreceptor cell; (b) one retinal pigmented epithelial cell; or (c) one Müller glial cell.

In one embodiment, a method is provided for identifying a bioactive agent that is capable of enhancing survival of a neuronal cell, comprising (i) contacting a candidate agent with the retinal neuronal cell culture system as described herein under conditions and for a time sufficient to permit interaction between a retinal neuronal cell of the retinal cell culture stress model system and the candidate agent; and (ii) comparing survival of a retinal neuronal cell of the cell culture system in the presence of the candidate agent with survival of a retinal neuronal cell of the cell culture stress system in the absence of the candidate agent, and therefrom identifying a bioactive agent that is capable of enhancing survival of the retinal neuronal cell. In certain particular embodiments, the retinal neuronal cell is a photoreceptor cell. In certain other particular embodiments, the retinal neuronal cell is a ganglion cell. In certain other embodiments, the retinal neuronal cell is a bipolar cell, a horizontal cell, or an amacrine cell.

In another embodiment, a method is provided for identifying a bioactive agent that is capable of inhibiting neurodegeneration of a retinal neuronal cell comprising (i) contacting a bioactive agent with a retinal neuronal cell culture stress model system as described herein, under conditions and for a time sufficient to permit interaction between a retinal neuronal cell of the cell culture stress model system and the candidate agent; and (ii) comparing structure of a retinal neuronal cell of the cell culture stress model system in the presence of the bioactive agent with structure of a retinal neuronal cell of the cell culture stress model system in the absence of the bioactive agent, and therefrom identifying a bioactive agent that is capable of inhibiting neurodegeneration of the retinal neuronal cell. In certain particular embodiments, the retinal neuronal cell is a photoreceptor cell. In certain other particular embodiments, the retinal neuronal cell is a ganglion cell. In certain other embodiments, the retinal neuronal cell is a bipolar cell, a horizontal cell, or an amacrine cell

In one embodiment, a method is provided for identifying a retinal cell stressor comprising (i) contacting a candidate stressor with a retinal cell culture stress model system comprising a first stressor as described herein, under conditions and for a time sufficient to permit interaction between a retinal cell of the cell culture stress model system and the candidate stressor; and (ii) comparing structure of a retinal cell of the cell culture stress model system in the presence of the candidate stressor with structure of a retinal cell of the cell culture stress model system in the absence of the candidate stressor, and therefrom identifying a retinal cell stressor that is capable of altering viability of a retinal cell, altering neurodegeneration of the retinal neuronal cell, or altering survival of a retinal cell. In certain particular embodiments, the retinal neuronal cell is a photoreceptor cell. In certain other particular embodiments, the retinal neuronal cell is a ganglion cell. In other particular embodiments, the candidate stressor increases neurodegeneration of the retinal neuronal cell. In certain other embodiments, the method comprises comparing survival of a retinal cell of the cell culture stress model system in the presence of the candidate stressor with survival of a retinal cell of the cell culture stress model system in the absence of the candidate stressor, and therefrom identifying a retinal cell stressor that is capable of altering survival of the retinal cell. In a particular embodiment, the stressor decreases or impairs survival of a retinal cell.

The invention also provides a method for identifying a bioactive agent that is capable of treating a retinal disease comprising contacting a bioactive agent with a retinal cell culture stress model system as described herein, under conditions and for a time sufficient to permit interaction between a retinal neuronal cell of the cell culture stress model system and the candidate agent; and (ii) comparing neurodegeneration of a retinal neuronal cell of the cell culture stress model system in the presence of the bioactive agent with neurodegeneration of a retinal neuronal cell of the cell culture stress model system in the absence of the bioactive agent, and therefrom identifying a bioactive agent that is capable of treating a retinal disease. In certain specific embodiments the retinal disease that is treated is macular degeneration, glaucoma, diabetic retinopathy, retinal detachment, retinal blood vessel occlusion, retinitis pigmentosa, optic neuropathy, inflammatory retinal disease, or a retinal disorder associated with Alzheimer's disease, Parkinson's disease, or multiple sclerosis. In certain specific embodiments, the retinal disease that is treated is the dry form of macular degeneration. In certain other specific embodiments, the retinal disease that is treated is glaucoma.

In another embodiment, a method is provided for identifying a bioactive agent that alters survival of a retinal neuronal cell, wherein the method comprises (1) contacting a candidate bioactive agent and a cell culture system comprising mature retinal cells and at least one cell stressor under conditions and for a time sufficient to permit interaction between a retinal neuronal cell and the candidate bioactive agent and (2) comparing survival of a retinal neuronal cell in the presence of the candidate bioactive agent with survival of a retinal neuronal cell in the absence of the candidate agent, thereby identifying a bioactive agent that is capable of altering survival of a retinal neuronal cell. In one embodiment, the cell stressor is selected from light, A2E, cigarette smoke condensate, increased atmospheric pressure, and glutamate. In a particular embodiment, the cell stressor is cigarette smoke condensate. In another embodiment, the method comprises at least two cell stressors selected from light, A2E, cigarette smoke condensate, increased atmospheric pressure, and glutamate. In a certain embodiment, two cell stressors are light and cigarette smoke condensate.

In specific embodiments the cell culture system excludes addition of other cells such as purified glial cells or ciliary body cells. In another embodiment, the retinal cell culture system comprises mature peripheral retinal cells derived from the anterior retina, and in certain other embodiments the cell culture system comprises mature retinal cells derived from the posterior retina. In another embodiment the cell culture system comprising mature retinal cells comprises extended culture of photoreceptors. In certain specific embodiments, the cultured photoreceptors have an intact outer segment.

In another embodiment, the invention provides methods for producing the retinal cell culture stress model comprising producing the cell culture system of mature retinal cells and adding a retinal cell stressor. In a particular embodiment, the method provides a retinal cell culture system comprising retinal cells that does not require the addition of other types of cells such as purified glia or cells isolated from a ciliary body or other part of the eye.

These and other embodiments of the invention will become evident upon reference to the following detailed description and attached drawings. In addition, references set forth herein that describe in more detail certain embodiments of this invention are therefore incorporated by reference in their entireties. All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

### BRIEF DESCRIPTION OF THE DRAWINGS

FIG. 1 provides a schematic of a photoreceptor cell.

FIG. 2 illustrates immunohistochemical staining of adult retinal cells. Porcine retinal cells were cultured for 1 week (FIG. 2A, 2B, 2C); 3 weeks (FIG. 2D, 2E, 2F); 6 weeks (FIG. 2G, 2H, 2K); and 8 weeks (FIG. 2J, 2K, 2L). Cells were subjected to an immunological analysis using a rhodopsin antibody to identify photoreceptors (FIG. 2A, 2D, 2G, 2J); an NFM antibody to identify ganglion cells (FIG. 2B, 2E, 2H, 2K); and an antibody to calretinin to identify amacrine and horizontal cells (FIG. 2C, 2F, 21, 2L).

FIG. 3 presents histograms showing the number of rhodopsin-expressing photoreceptors after no stress or white light stress, demonstrating a dose response to both duration (FIG. 3A) and intensity (FIG. 3B).

FIG. 4 presents a histogram showing the number of NFM-expressing ganglion cells after no stress or 6000 lux of white light stress for 24 hours.

FIG. 5 presents a histogram showing the number of TUNEL-positive nuclei after 24 hours of no stress or 6000 lux white light stress followed by a 13-hour rest period.

FIG. 6 presents a histogram showing the number of rhodopsin-expressing photoreceptors after no stress or 2000 lux of blue light stress for varying times followed by a 14 hour rest period.

FIG. 7 presents data showing the number of rhodopsin-expressing photoreceptors after 24 hours of no stress or A2E stress at varying concentrations.

FIG. 8 presents a histogram showing the number of NFM-expressing ganglion cells after 24 hours of no stress or 20 μM A2E stress.

FIG. 9 shows the effect of cigarette smoke condensate stress (100 μg/ml) on rhodopsin-expressing photoreceptors.

FIG. 10 depicts the effect on rhodopsin-expressing photoreceptors of no stress and on photoreceptors under white light stress (1500 lux) plus cigarette smoke condensate stress (100 μg/ml).

FIGS. 11A-11D illustrate the effect of increased atmospheric pressure stress on cultured mature retinal neuronal cells. FIGS. 11A and 11B show representative ganglion cells that were not exposed to increased atmospheric pressure (75 mm Hg) as a stressor. FIGS. 11C and 11D illustrate examples of apoptotic ganglion cells. Apoptotic ganglion cells detected with an anti-caspase-3 antibody are indicated by arrows.

FIG. 12 shows immunohistochemical analysis of representative rhodopsin-expressing photoreceptors before stress.

FIG. 13 illustrates immunohistochemical analysis of representative rhodopsin-expressing photoreceptors after stress (25 μM A2E for 24 hours). The small dots indicate cell debris.

FIG. 14 shows an immunohistochemical analysis of rhodopsin-expressing photoreceptors under stress but with addition of EPO (1 U/mL) for 24 hours.

### DETAILED DESCRIPTION OF THE INVENTION

The present invention relates to the discovery of a cell culture model that comprises a long-term or extended culture of mature retinal cells, including retinal neuronal cells (e.g., photoreceptor cells, amacrine cells, ganglion cells, horizontal cells, and bipolar cells). Surprisingly, the cell culture system and methods for producing the cell culture system provide extended culture of photoreceptor cells. The cell culture system described herein may also comprise retinal pigmented epithelial (RPE) cells and Müller glial cells.

In one embodiment, the retinal cell culture system comprises a cell stressor. The application or the presence of the stressor affects the mature retinal cells, including the retinal neuronal cells, in vitro in a manner that is useful for studying disease pathology that is observed in a retinal disease or disorder. The cell culture model described herein provides an in vitro neuronal cell culture system that will be useful in the identification and biological testing of new neuroactive compounds or bioactive agents that may be suitable for treatment of neurological diseases or disorders in general, and for treatment of degenerative diseases of the eye and brain in particular. The ability to obtain primary cells from mature, fully-differentiated retinal cells, including retinal neurons for culture in vitro over an extended period of time in the presence of a stressor enables examination of cell-to-cell interactions, selection and analysis of neuroactive compounds and materials, use of a controlled cell culture system for in vivo CNS and ophthalmic tests, and analysis of the effects on single cells from a consistent retinal cell population.

The cell culture system described herein and the retinal cell stress model comprising cultured mature retinal cells, retinal neurons, and a retinal cell stressor are particularly useful for candidate compound screening to identify bioactive agents capable of inducing or stimulating regeneration of CNS tissue that has been damaged by disease. In certain embodiments, the cultured mature retinal neurons comprise all the major retinal neuronal cell types including photoreceptor, amacrine, ganglion cells, horizontal cells, and bipolar cells. The retinal cell culture system described herein comprising one or more cell stressors presents a needed alternative to expensive and time-consuming in vivo animal models for studying the pathology and progression of retinal diseases and disorders and for identifying therapeutic agents for treatment of these diseases.

Identifying bioactive agents may be useful for treating, curing, preventing, ameliorating the symptoms of, or slowing, inhibiting, or stopping the progression of a neurodegenerative disease or disorder. Such neurodegenerative diseases include but are not limited to glaucoma, macular degeneration, diabetic retinopathy, retinal detachment, retinal blood vessel (artery or vein) occlusion, retinitis pigmentosa, optic neuropathy, inflammatory retinal disease, and retinal disorders associated with other neurodegenerative diseases such as Alzheimer's disease, multiple sclerosis, or Parkinson's Disease, or associated with AIDS. Long-term or extended cell culture of photoreceptor cells in particular is useful for identifying agents that will be useful for treating retinal diseases and disorders that are characterized by photoreceptor neurodegeneration such as the dry form of macular degeneration, and those that are characterized by ganglion cell neurodegeneration such as glaucoma.

Retinal Cells

The in vitro cell culture system described herein permits and promotes the survival in the culture of mature retinal cells, including retinal neurons, for at least 2-4 weeks, over 2 months, or for as long as 6 months. Retinal cells are isolated from non-embryonic, non-tumorigenic tissue and have not been immortalized by any method such as, for example, transformation or infection with an oncogenic virus. The cell culture system may comprise all the major retinal neuronal cell types (photoreceptors, bipolar cells, horizontal cells, amacrine cells, and ganglion cells), and also may include other mature retinal cells such as retinal pigmented epithelial cells and Müller glial cells.

The retina of the eye is a thin, delicate layer of nervous tissue. The major landmarks of the retina are the area centralis in the posterior portion of the eye and the peripheral retina in the anterior portion of the eye. The retina is thickest near the posterior sections and becomes thinner near the periphery. The area centralis is located in the posterior retina and contains the fovea and foveola and, in primates, contains the macula. The foveola contains the area of maximal cone density and, thus, imparts the highest visual acuity in the retina. The foveola is contained within the fovea, which is contained within the macula.

The peripheral or anterior portion of the retina increases the field of vision. The peripheral retina extends anterior to the equator of the eye and is divided into four regions: the near periphery (most posterior), the mid-periphery, the far periphery, and the ora serrata (most anterior). The ora serrata denotes the termination of the retina.

The term neuron (or nerve cell) as understood in the art and used herein denotes a cell that arises from neuroepithelial cell precursors. Mature neurons (i.e., fully differentiated cells from an adult) display several specific antigenic markers. Neurons may be classified functionally into three groups: (1) afferent neurons (or sensory neurons) that transmit information into the brain for conscious perception and motor coordination; (2) motor neurons that transmit commands to muscles and glands; and (3) intemeurons that are responsible for local circuitry; and (4) projection intemeurons that relay information from one region of the brain to anther region and therefore have long axons. Intemeurons process information within specific subregions of the brain and have relatively shorter axons. A neuron typically has four defined regions: the cell body (or soma); an axon; dendrites; and presynaptic terminals. The dendrites serve as the primary input of information from other cells. The axon carries the electrical signals that are initiated in the cell body to other neurons or to effector organs. At the presynaptic terminals, the neuron transmits information to another cell (the postsynaptic cell), which may be another neuron, a muscle cell, or a secretory cell.

The retina is composed of several types of neuronal cells. As described herein, the types of retinal neuronal cells that may be cultured in vitro by this method include photoreceptor cells, ganglion cells, and interneurons such as bipolar cells, horizontal cells, and amacrine cells. Photoreceptors are specialized light-reactive neural cells and comprise two major classes, rods and cones. Rods are involved in scotopic or dim light vision, whereas photopic or bright light vision originates in the cones by the presence of trichromatic pigments. Many neurodegenerative diseases that result in blindness, such as macular degeneration, retinal detachment, retinitis pigmentosa, diabetic retinopathy, etc, affect photoreceptors.

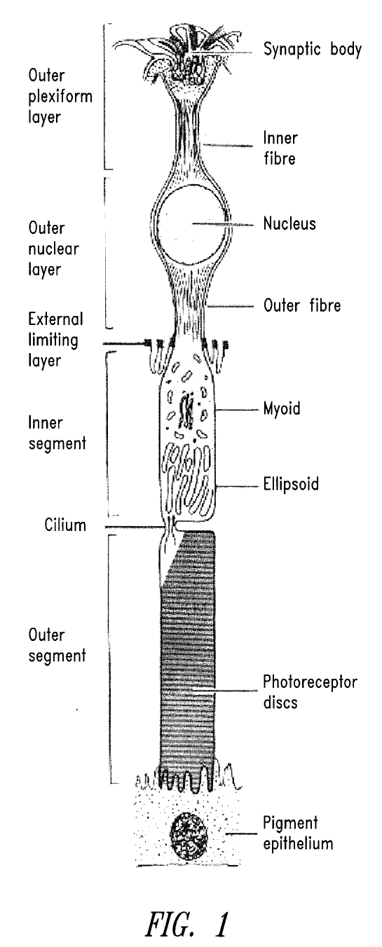

Extending from their cell bodies, the photoreceptors have two morphologically distinct regions, the inner and outer segments (see FIG. 1). The outer segment lies furthermost from the photoreceptor cell body and contains disks that convert incoming light energy into electrical impulses (phototransduction). As shown in FIG. 1, the outer segment is attached to the inner segment with a very small and fragile cilium. The size and shape of the outer segments vary between rods and cones and are dependent upon position within the retina. See Eye and Orbit, 8^{th} Ed., Bron et al., (Chapman and Hall, 1997).

Ganglion cells are output neurons that convey information from the retinal interneurons (including horizontal cells, bipolar cells, amacrine cells) to the brain. Bipolar cells are named according to their morphology, and receive input from the photoreceptors, connect with amacrine cells, and send output radially to the ganglion cells. Amacrine cells have processes parallel to the plane of the retina and have typically inhibitory output to ganglion cells. Amacrine cells are often subclassified by neurotransmitter or neuromodulator or peptide (such as calretinin or calbindin) and interact with each other, with bipolar cells, and with photoreceptors. Bipolar cells are retinal interneurons that are named according to their morphology; bipolar cells receive input from the photoreceptors and sent the input to the ganglion cells. Horizontal cells modulate and transform visual information from large numbers of photoreceptors and have horizontal integration (whereas bipolar cells relay information radially through the retina).

Other retinal cells that may be present in the retinal cell cultures described herein include glial cells, such as Müller glial cells, and retinal pigmented epithelial cells (RPE). Glial cells surround nerve cell bodies and axons. The glial cells do not carry electrical impulses but contribute to maintenance of normal brain function. Müller glia, the predominant type of glial cell within the retina, provide structural support of the retina and are involved in the metabolism of the retina (e.g., contribute to regulation of ionic concentrations, degradation of neurotransmitters, and remove certain metabolites (see, e.g., Kljavin et al., J. Neurosci. 11:2985 (1991)). Müller's fibers (also known as sustentacular fibers of retina) are sustentacular neuroglial cells of the retina that run through the thickness of the retina from the internal limiting membrane to the bases of the rods and cones where they form a row of junctional complexes.

Retinal pigmented epithelial (RPE) cells form the outermost layer of the retina, nearest the blood vessel-enriched choroids. RPE cells are a type of phagocytic epithelial cell, functioning like macrophages, that lies below the photoreceptors of the eye. The dorsal surface of the RPE cell is closely apposed to the ends of the rods, and as discs are shed from the rod outer segment they are internalized and digested by RPE cells. RPE cells also produce, store, and transport a variety of factors that contribute to the normal function and survival of photoreceptors. Another function of RPE cells is to recycle vitamin A as it moves between photoreceptors and the RPE during light and dark adaptation.

Cell Culture System

In one embodiment, a cell culture system is provided that comprises a plurality of mature retinal cell, wherein the cell culture system is substantially free of cells purified from a non-retinal tissue source. The cell culture system disclosed herein differs from previously reported systems in that overall mature retinal cell survival, including retinal neuronal cell survival, and in particular, photoreceptor survival, is robust over time without the express addition of other non-retinal cell types such as ciliary body cells, or purified stem cells or purified glia (i.e., stem cells or glial cells isolated and purified separately from a non-retinal tissue or other source). Combinations of factors, such as ciliary neurotrophic factor (CNTF), brain-derived neurotrophic factor (BDNF), fibroblast growth factor-2 (FGF2), and glial cell line-derived neurotrophic factor (GDNF) also have been reported to improve the survival of photoreceptors in organ cell culture systems (Oglivie et al., Exp. Neurol. 161:676-85 (2000)), but none of these factors sustain survival of neuronal cells in these reported culture systems for long periods of time. Others groups have reported in vitro culture of embryonic retinal neurons, but the cultured embryonic retinal cells either failed to express all of the retina-specific proteins that are expressed by mature retinal cells or these cells could only be cultured for short times.

The in vitro cell culture system described herein permits and promotes (or extends) the survival in culture of mature retinal cells, including retinal neurons, for over 2 months and for as long as 6 months. Until now, the ability to screen drug candidates using mature retinal cells has been limited to the life span of the retinal cells (between one and two weeks), including retinal neurons, in primary culture. See also, e.g., Luo et al., Invest. Ophthalmol. Vis. Sci. 42:1096-1106 (2001); Gaudin et al., Invest. Ophthalmol. Vis. Sci. 37:2258-68 (1996). Delays in enucleation and delays in tissue dissociation have a severe deleterious effect on recovery and survival of neurons (see, for example, Gaudin et al., supra). Neurons begin to deteriorate immediately after being dissociated from the animal body, and the resulting deterioration precludes adequate and reliable compound screening to identify agents that may be used for treating retinal diseases. Also, without the ability to maintain a long-term retinal cell culture, performing various analyses related to either projection neurons or photoreceptor cells is difficult. Photoreceptors are the primary cell type affected in macular degeneration, a leading cause of blindness. Ganglion cells, projection neurons in the retina, are affected in glaucoma patients, also a leading cause of blindness.

The cell culture system described herein comprises the culture of retinal cells including retinal neurons in vitro for extended periods of time, thus providing viable, fully mature retinal cells and neurons for a period greater than 2 months. Also provided herein is a method for producing the cell culture system comprising isolating mature retinal cells from a biological source and culturing the mature retinal cells under conditions that maintain viability of the mature retinal cells. Viability of the retinal cells in the cell culture system means that all or a portion of the cells that are isolated and plated for tissue culture as described herein metabolize and exhibit structure and functions of a healthy, thriving cell that is characteristic for the particular cell type. Viability of one or more of the mature retinal cell types is maintained for an extended period of time, for example, at least 4 weeks, 2 months (8 weeks), or at least 4-6 months, for at least 10%, 25%, 40%, 50%, 60%, 70%, 80%, or 90% of the mature retinal cells that are isolated (harvested) from retinal tissue and plated for tissue culture. Viability of the retinal cells may be determined according to methods described herein and known in the art. Retinal neuronal cells, similar to neuronal cells in general, are not actively dividing cells in vivo and thus cell division of retinal neuronal cells would not necessarily be indicative of viability. An advantage of the cell culture system is the ability to culture amacrine cells, photoreceptors and associated ganglion projection neurons for extended periods of time, thereby providing an opportunity to screen for compounds that will be effective for treatment of retinal disease.

The disclosed methods and cell culture systems may also be applicable to brain and spinal cord diseases. A chronic disease model is of particularly importance because most neurodegenerative diseases are chronic. In addition, through use of this in vitro cell culture system, the earliest events in long-term disease development processes may be identified because an extended period of time is available for cellular analysis. The long-term mature retinal culture system described herein also is useful for experiments that are relatively short term in duration (e.g., 3-14 days) because the baseline for survival and viability is more stable than in short-term culture models heretofore developed in which the cells are progressively dying.

The cell culture system described herein provides a mature retinal cell culture that is a mixture of mature retinal neuronal cells and non-neuronal retinal cells. The cell culture system may comprise all the major retinal neuronal cell types (photoreceptors, bipolar cells, horizontal cells, amacrine cells, and ganglion cells), and also includes other mature retinal cells such as RPE and Müller glial cells. By incorporating these different types of cells into the in vitro culture system, the system essentially resembles an “artificial organ” that is more akin to the natural in vivo state of the retina.

The mature retinal cells and retinal neurons may be cultured in vitro for extended periods of time, longer than 2 days or 5 days, longer than 2 weeks, 3 weeks, or 4 weeks, and longer than 2 months (8 weeks), 3 months (12 weeks), and 4 months (16 weeks), and longer than 6 months, thus providing a long-term culture. In certain embodiments, at least 20-40%, at least 50%, at least 60%, at least 70%, at least 80%, or at least 90% of one or more of the mature retinal cell types remain viable in this long-term cell culture system. The biological source of the retinal cells or retinal tissue may be mammalian (e.g., human, non-human primate, ungulate, rodent, canine, porcine, bovine, or other mammalian source), avian, or from other genera. In one embodiment, retinal cells including retinal neurons from post-natal non-human primates, post-natal pigs, or post-natal chickens may be used, but any adult or post-natal retinal tissue may be suitable for use in this retinal cell culture system. The types of retinal neuronal cells that may be cultured in vitro by this method include ganglion cells, photoreceptors, bipolar cells, horizontal cells, and amacrine cells. Non-neuronal retinal cells that are cultured with the retinal neurons are cells that are derived from the original retinal tissue, and include, for example, RPE cells and Müller glial cells.

The cell culture system described herein provides for robust long-term survival of retinal cells without inclusion of cells derived from or isolated or purified from non-retinal tissue. The cell culture system comprises cells isolated solely from the retina of the eye and thus is substantially free of types of cells from other parts or regions of the eye that are separate from the retina, such as ciliary bodies and vitreous. A retinal cell culture that is substantially free of non-retinal cells contains retinal cells that comprise preferably at least 80-85% of the cell types in culture, preferably 90%-95%, or preferably 96%-100% of the cell types. Retinal cells in the cell culture system are viable and survive in the cell culture system without added purified (or isolated) glial cells or stem cells from a non-retinal source, or other non-retinal cells. As described herein the retinal cell culture system is prepared from isolated retinal tissue only, thereby rendering the cell culture system substantially free of non-retinal cells.

Persons skilled in the cell culture art appreciate that successfully obtaining a long-term or extended culture of cells derived directly from a tissue source (i.e., a primary cell culture) and maintaining viability of the cells (e.g., retinal cells) in culture depends on several factors. Similar to establishing a long-term culture of any tissue-derived cell population (even including tumor tissue for propagation of immortalized cancer cells), the length of time that passes between harvesting of a retinal tissue and plating of the cells can particularly affect successful establishment of a long term culture. Neurons begin to deteriorate immediately after being dissociated from neural tissue. Delays in enucleation and delays in tissue dissociation have a severe deleterious effect on recovery and survival of neurons (see, for example, Gaudin et al, supra).

Accordingly, methods for producing an extended retinal cell culture may benefit from minimizing the time periods between harvesting the tissue (which also includes minimizing the time between the death of the source animal and when the tissue is harvested) and dissecting the tissue, and the time between initiation and completion of the dissection and dissociation procedures and plating of the cells. For example, in preparation of the retinal cell culture, the eyes that are dissected are preferably obtained and dissected within 12 hours of harvesting the organ. In addition, the dissection methods described herein are performed more quickly than previously described methods for culturing retinal cells. The efficiency of this method is improved over methods for production of other retinal cell culture systems that combine retinal cells with other cell types from the eye or other regions of the CNS, by eliminating those additional cell preparation steps. Other factors that can affect successful culturing of tissue-derived cells include the temperature at which the tissues are maintained during and after transport, the health and age of the tissue donor, the skill of the animal handler, surgeon, and/or cell culturist, and similar factors appreciated by those skilled in the art.

Dissection of the eye may be performed according to standard procedures known in the art and described herein. By way of example, eyes obtained from a donor animal are enucleated, and muscle and other tissue are cleaned away from the eye orbit. In one embodiment, the peripheral retina is dissected from other portions or regions of the eye. The eyes are cut in half along their equator, and the neural retina is dissected from the anterior part of the eye. The retina, ciliary body, and vitreous are dissected away from the anterior half portion of the eye in a single piece, followed by gentle detachment of the opaque retina from the clear vitreous. In another embodiment, the posterior portion of the retina containing the area centralis is isolated from other regions of the eye by dissection. The posterior portion of the retina contains the fovea (and the macula in primates), with a higher concentration of cone photoreceptors, whereas the anterior portion of the retina has a higher concentration of rod photoreceptors. Pigmented epithelial cells may or may not be totally separated from the dissected retina.

Retinal cells may be isolated from retinal tissue by mechanical means, such as dissection and teasing (trituration). Tissues of the eye may also be treated with one or more enzymes including but not limited to papain, hyaluronidase, collagenase, trypsin, and/or a deoxyribonuclease, to dissociate the cells and remove undesired cellular components. The cell culture system may be prepared by a combination of mechanical methods and enzymatic digestion.

The cell culture systems and methods described herein may employ use of any plastic or glass surface (including, for instance, coverslips), preferably surfaces that are manufactured for cell culture use for providing a surface to which the retinal cells can adhere. The surface may also be coated with an attachment-enhancing substance or a combination of such substances, such as poly-lysine, Matrigel, laminin, polyomithine, gelatin, and/or fibronectin, or the like. Retinal cells prepared from an eye as described herein may be plated onto one surface, such as a glass coverslip, which is then placed in a tissue culture container and immersed in tissue culture media. The tissue culture container may be, for example, a multi-well plate such as a 24-well tissue culture plate. Alternatively, one or more surfaces onto which the retinal cells are plated (and to which the cells will adhere) may be placed in one or more tissue culture flasks, which are familiar to persons in the art. Alternatively, the retinal cells may be applied to and maintained in standard tissue culture multi-well dishes and/or tissue culture flasks. Feeder cell layers, such as glial feeder layers, epithelial cell layers, or embryonic fibroblast feeder layers, may also find use within the methods and systems provided herein.

For maintaining viability of the retinal cells in the cell culture system, the system also comprises components and conditions known in the art for proper maintenance of cells in culture, including media (with or without antibiotics) that contains buffers and nutrients (e.g., glucose, amino acids (e.g., glutamine), salts, minerals (e.g., selenium)) and also may contain other additives or supplements (e.g., fetal bovine serum or an alternative formulation that does not require a serum supplement; transferrin; insulin; putrescine; progesterone) that are required or are beneficial for in vitro culture of cells and that are well known to a person skilled in the art (see, for example, Gibco media, Invitrogen Life Technologies, Carlsbad, Calif.). Similar to standard cell culture methods and practices, the retinal cell cultures described herein are maintained in tissue culture incubators designed for such use so that the levels of carbon dioxide, humidity, and temperature can be controlled. The cell culture system may also comprise addition of exogenous (i.e., not produced by the cultured cells themselves) cell growth factors or neurotrophic factors, which may be provided, for example, in the media or in the substrate or surface coating.

Retinal Neuronal Cell Culture Stress Model

The in vitro retinal cell culture systems described herein may serve as a physiological retinal model that can be used to characterize the physiology of the retina. This physiological retinal model may also be used as a broader general neurobiology model. A cell stressor may be included in the model cell culture system. A cell stressor, which as described herein is a retinal cell stressor, adversely affects the viability or reduces the viability of one or more of the different retinal cell types in the culture, including types of retinal neuronal cells, in the cell culture system. A person skilled in the art would readily appreciate and understand that as described herein a retinal cell which exhibits reduced viability means that the length of time that a retinal cell survives in the cell culture system is reduced or decreased (decreased lifespan) and/or that the retinal cell exhibits a decrease, inhibition, or adverse effect of a biological or biochemical function (decreased or abnormal metabolism; initiation of apoptosis; etc.) compared with a retinal cell cultured in an appropriate control cell system (e.g., the cell culture system described herein in the absence of the cell stressor). Reduced viability of a retinal cell may be indicated by cell death; an alteration or change in cell structure or morphology; induction and/or progression of apoptosis; initiation, enhancement, and/or acceleration of retinal neuronal cell neurodegeneration (or neuronal cell injury).

Methods and techniques for determining cell viability are described in detail herein and are those with which skilled artisans are familiar. These methods and techniques for determining cell viability may be used for monitoring the health and status of retinal cells in the cell culture system described herein, for identifying cell stressors that reduce retinal cell viability and, as also described herein, for identifying a bioactive agent that alters (preferably increases) retinal cell viability.

The addition of a cell stressor to the cell culture system described herein may be used to identify bioactive agents that abrogate, inhibit, eliminate, or lessen the effect of the stressor. The retinal neuronal cell culture system may include a cell stressor that is chemical (e.g., A2E, cigarette smoke concentrate), biological (for example, toxin exposure; beta-amyloid; lipopolysaccharides), or non-chemical, such as a physical stressor, environmental stressor, or a mechanical force (e.g., increased pressure or light exposure).

The retinal cell stressor model system may also include a cell stressor such as, but not limited to, a stressor that may be a risk factor in a disease or disorder or that may contribute to the development or progression of a disease or disorder, including but not limited to, light of varying wavelengths and intensities; cigarette smoke condensate exposure; glucose oxygen deprivation; oxidative stress (e.g., stress related to the presence of or exposure to hydrogen peroxide, nitroprusside, Zn++, or Fe++); increased pressure (e.g., atmospheric pressure or hydrostatic pressure), glutamate or glutamate agonist (e.g., N-methyl-D-aspartate (NMDA); alpha-amino-3-hydroxy-5-methylisoxazole-4-proprionate (AMPA); kainic acid; quisqualic acid; ibotenic acid; quinolinic acid; aspartate; trans-1-aminocyclopentyl-1,3-dicarboxylate (ACPD)); amino acids (e.g., aspartate, L-cysteine; beta-N-methylamine-L-alanine); heavy metals (such as lead); various toxins (for example, mitochondrial toxins (e.g., malonate, 3-nitroproprionic acid; rotenone, cyanide); MPTP (1-methyl-4-phenyl-1,2,3,6,-tetrahydropyridine), which metabolizes to its active, toxic metabolite MPP+ (1-methyl-4-phenylpryidine)); 6-hydroxydopamine; alpha-synuclein; protein kinase C activators (e.g., phorbol myristate acetate); biogenic amino stimulants (for example, methamphetamine, MDMA (3-4 methylenedioxymethamphetamine)); or a combination of one or more stressors. Useful retinal cell stressors include those that mimic a neurodegenerative disease that affects any one or more of the mature retinal cells described herein. A chronic disease model is of particular importance because most neurodegenerative diseases are chronic. Through use of this in vitro cell culture system, the earliest events in long-term disease development processes may be identified because an extended period of time is available for cellular analysis.

In certain embodiments, the methods described herein may be used for identifying a cell stressor that alters viability (i.e., alters survival and/or neurodegeneration and/or neuronal cell injury) of one, two, three, or more, or all retinal cell types and may also be used to identify a stressor that alters viability of one, two, three, or more or all retinal neuronal cell types (amacrine cell, a photoreceptor cell, a ganglion cell, horizontal cell, and bipolar cell). In certain other embodiments, the screening methods may be used to identify a cell stressor that alters viability (preferably decreases survival and/or promotes or enhances neurodegeneration or cell injury) of one retinal neuronal cell type, such as an amacrine cell, a photoreceptor cell, a ganglion cell, a horizontal cell, or a bipolar cell.

A retinal cell stressor may alter (i.e., increase or decrease in a statistically significant manner) viability of retinal cells such as by altering survival of retinal cells, including retinal neuronal cells, or by altering neurodegeneration of retinal neuronal cells. Preferably, a retinal cell stressor adversely affects a retinal neuronal cell such that survival of a retinal neuronal cell is decreased or adversely affected (i.e., the length of time during which the cells are viable is decreased in the presence of the stressor) or neurodegeneration (or neuron cell injury) of the cell is increased or enhanced. The stressor may affect only a single retinal cell type in the retinal cell culture or the stressor may affect two, three, four, or more of the different cell types. For example, a stressor may alter viability and survival of photoreceptor cells but not affect all the other major cell types (e.g., ganglion cells, amacrine cells, horizontal cells, bipolar cells, RPE, and Müller glia). Stressors may shorten the survival time of a retinal cell (in vivo or in vitro), increase the rapidity or extent of neurodegeneration of a retinal cell, or in some other manner adversely affect the viability, morphology, maturity, or lifespan of the retinal cell.

The effect of a cell stressor on the viability of retinal cells in the cell culture system may be determined for one or more of the different retinal cell types. Determination of cell viability may include evaluating structure and/or a function of a retinal cell continually at intervals over a length of time or at a particular time point after the retinal cell culture is prepared. Viability or long term survival of one or more different retinal cell types or one or more different retinal neuronal cell types may be examined according to one or more biochemical or biological parameters that are indicative of reduced viability, such as apoptosis or a decrease in a metabolic function, prior to observation of a morphological or structural alteration.

A chemical, biological, or physical cell stressor may reduce viability of one or more of the retinal cell types present in the cell culture system when the stressor is added to the cell culture under conditions described herein for maintaining the long-term cell culture. Alternatively, one or more culture conditions may be adjusted so that the effect of the stressor on the retinal cells can be more readily observed. For example, the concentration or percent of fetal bovine serum may be reduced or eliminated from the cell culture when cells are exposed to a particular cell stressor. When a serum-free media is desired for a particular purpose, cells may be gradually weaned (i.e., the concentration of the serum is progressively and often systematically decreased) from an animal source of serum into a media that is free of serum or that contains a non-serum substitute. The decrease in serum concentration and the time period of culture at each decreased concentration of serum may be continually evaluated and adjusted to ensure that cell survival is maintained. When the retinal cell culture system described herein is exposed to a cell stressor, the serum concentration may be adjusted concomitantly with the application of the stressor (which may also be titrated (if chemical or biological) or adjusted (if a physical stressor)) to achieve conditions such that the stress model is useful for evaluating the effect of the stressor on a retinal cell type and/or for identifying an agent that inhibits, reduces, or abrogates the adverse effect(s) of a stressor on the retinal cell. Alternatively, retinal cells cultured in media containing serum at a particular concentration for maintenance of the cells may be abruptly exposed to media that does not contain any level of serum. In another embodiment, serum may be decreased in a retinal cell culture to less than 5%, 2%, 1%, 0.5%, less than 0.25%, less than 0.1%, or less than 0.05% in a single step.

The retinal cell culture may be exposed to a cell stressor for a period of time that is determined to reduce the viability of one or more retinal cell types in the retinal cell culture system. The length of time that the culture is exposed to a cell stressor may be 3 hours, 6 hours, 9 hours, 12, hours, 18 hours, 24 hours, or 2 days, 3 days, 4 days, 5 days, 6 days, or a week, at least two weeks, and at least one month, or longer, or for any period of time between the time periods enumerated. The cells may be exposed to a cell stressor immediately upon plating of the retinal cells after isolation from retinal tissue. Alternatively, the retinal cell culture may be exposed to a stressor after the culture is established, or any time thereafter (e.g., one day, two days, 3-5 days, 6-10 days, 2 weeks, 3 weeks, or 4 weeks). When two or more cell stressors are included in the retinal cell culture system, each stressor may be added to the cell culture system concurrently and for the same length of time or may be added separately at different time points for the same length of time or for differing lengths of time during the culturing of the retinal cell system.

Viability of the retinal cells in the cell culture system may be determined by any one or more of several methods and techniques described herein and practiced by skilled artisans (see also, e.g., methods and techniques described herein regarding determining viability in the presence of a bioactive agent). The effect of a stressor may be determined by comparing structure or morphology of a retinal cell, including a retinal neuronal cell, in the cell culture system in the presence of the stressor with structure or morphology of the same cell type of the cell culture system in the absence of the stressor, and therefrom identifying a stressor that is capable of altering neurodegeneration of the neuronal cell. The effect of the stressor on viability can also be evaluated by methods known in the art and described herein, for example by comparing survival of a neuronal cell of the cell culture system in the presence of the stressor with survival of a neuronal cell of the cell culture system in the absence of the stressor, and therefrom identifying a stressor that is capable of altering survival of the neuronal cell.

Survival of retinal cells may be determined according to methods described in detail herein and known in the art that identify and characterize retinal cells, for example, immunocytochemical methods. Antibodies that specifically bind to cell markers for a specific retinal or retinal neuronal cell type as well as antibodies that bind to cytoskeletal proteins common to more than one cell type are commercially available. Alternatively, such antibodies can be prepared according to standard methods and techniques known in the art (see, e.g., Kohler and Milstein, Eur. J. Immunol. 6:511-519 (1976) and improvements thereto; Harlow and Lane, Antibodies: A Laboratory Manual, Cold Spring Harbor Laboratory (1988); Antibody Engineering, Methods and Protocols, Lo, ed., (Human Press 2004); U.S. Pat. Nos. 5,693,762; 5,585,089; 4,816,567; 5,225,539; 5,530,101; U.S. Pat. No. 5,223,409; Schlebusch et al., Hybridoma 16:47 (1997); and references cited therein; see also Andris-Widhopf et al., J. Immunol. Methods 242:159-81 (2000)).

Photoreceptors may be identified using antibodies that specifically bind to photoreceptor-specific proteins such as opsins, peripherins, and the like. Photoreceptors in cell culture may also be identified as a morphologic subset of immunocytochemically labeled cells by using a pan-neuronal marker or may be identified morphologically in enhanced contrast images of live cultures. Outer segments can be detected morphologically as attachments to photoreceptors.

Retinal cells including photoreceptors can also be detected by functional analysis. For example, electrophysiology methods and techniques may be used for measuring the response of photoreceptors to light. Photoreceptors exhibit specific kinetics in a graded response to light. Calcium-sensitive dyes may also be used to detect graded responses to light within cultures containing active photoreceptors. For analyzing stress-inducing compounds or potential neurotherapeutics, retinal cell cultures can be processed for immunocytochemistry, and photoreceptors and/or other retinal cells can be counted manually or by computer software using photomicroscopy and imaging techniques. Other immunoassays known in the art (e.g., ELISA, immunoblotting, flow cytometry) may also be useful for identifying and characterizing the retinal cells and retinal neuronal cells of the cell culture model system described herein.

The retinal cell culture stress models may also be useful for identification of both direct and indirect pharmacologic agent effects. For example, certain candidate bioactive agents added to the cell culture system in the presence of one or more retinal cell stressors may stimulate one cell type in a manner that enhances or decreases the survival of other cell types. Cell/cell interactions and cell/extracellular component interactions may be important in understanding mechanisms of disease and drug function. For example, one neuronal cell type may secrete trophic factors that affect growth or survival of another neuronal cell type (see, e.g., WO 99/29279).

Light Stressor

In one embodiment, the retinal cell stressor is light. Light is believed to cause or contribute to retinal cell death, particularly photoreceptor cell death. Exposure to cumulative amounts of light is considered a risk factor for onset of macular degeneration. The results from animal studies have indicated that mice exposed to high intensity light develop similar pathophysiological effects as observed in humans with macular degeneration (see, e.g., Dithmar et al., Arch. Ophthalmol. 119:1643-49 (2001); Gottsch et al., Arch. Ophthalmol. 111:126-29 (1993)).

For culture of retinal cells exposed to a light stressor, the light may be emitted from at least one fluorescent light, incandescent light, or at least one light-emitting diode. The exposure may be intermittent or constant, and the duration of exposure may be varied. Alternatively, light stress may be applied as a light shock whereby cells at some point prior to or during cell culture may be protected from exposure to any light source and then exposed to a light stress.

The intensity of the light stress may be measured in lux, which is a measure of light output at a surface. The retinal cell culture described herein is preferably exposed to light (white or blue light) at any intensity or at any range of intensities from about 1 to 20,000 lux, at any intensity or any range of intensities between about 1000-15,000 lux, between about 1000-8000 lux, between about 250-8000 lux, 250-1000 lux, 250-2000 lux, 250-4000 lux, between about 4000-8000 lux, between about 1000-6000 lux, between about 1000-4000, between about 2000-6000, between about 2000-4000, between about 4000-6000 lux, or between about 1000-2000 lux. In one embodiment, cells are exposed to moderate intensity, for example, about 4000-6000 lux over a short period of time, for example, less than one week, between 18-96 hours, or between 18-48 hours. In another embodiment, the retinal cells are exposed to lower intensity of light (for example, between about 500-4000 lux, or between about 500-2000 lux, between about 250-1000, or between about 500-1000 lux) over a longer period of time (such as, longer than one week, at least two weeks, or at least one month). The latter set of conditions (lower intensity of light over a longer period of time) may provide a stress model for evaluating the effect of stress in chronic neurodegenerative retinal diseases and for identifying bioactive agents that may be useful for treating chronic neurodegenerative retinal diseases.

The light stress may comprise ultraviolet or visible light at any wavelength varying from between 100 to 700 nm. In one embodiment, the light stress is visible light and may include light at any wavelength from approximately 400 nm (violet light) to approximately 700 nm (red light) of the electromagnetic spectrum. In certain embodiments, the light stress is blue light in the visible spectrum from approximately 425 nm to 500 nm, for example, 470 nm. The ultraviolet part of the spectrum (up to approximately 300-400 nm) is divided into three regions: the near ultraviolet, the far ultraviolet, and the extreme ultraviolet. The three regions are distinguished by how energetic the ultraviolet radiation is and by the wavelength of the ultraviolet light, which is related to energy. The near ultraviolet is the light closest to optical or visible light. The extreme ultraviolet is the ultraviolet light closest to X-rays, and is the most energetic of the three types. The far ultraviolet lies between the near and extreme ultraviolet regions.

The source of light may be a fluorescent light, incandescent light, or a light-emitting diode (LED); the light source may be inserted into a tissue culture incubator to provide continuous exposure or to regulate exposure during the time that the retinal cells are cultured. High intensity light sources are useful, providing the capability to apply light at variable intensity levels. In one embodiment, LED fixtures are designed to provide light stress to the cell cultures from above the cell culture plate (which may be any cell culture dish, flask, or multi-well plate) from one LED and below the cell culture plate from a second separate LED. Each LED may emit light of the same intensity or of different intensities, which may be controlled for example by different potentiometers to independently control the current flowing through each LED. The emitted light may be constant, that is, having the same wavelength and intensity over a period of time, or may be cyclical, varying the wavelength or intensity. For example, emitted light that is cyclical may be controlled such that the light stress mimics or matches a circadian rhythm. Light sources that are mounted in a tissue culture incubator can be appropriately placed to ensure proper ventilation such that exposure of the cells to the light source does not result in exposure of the cells or a portion of the cells to changes in temperature.

In another embodiment, the source of light is a fluorescent light fixture, for example, a set of linear bulbs to provide ambient light to an entire plate, flask, or dish of cells. The bulb may also be large enough to permit exposure of multiple cell culture plates, dishes, or flasks.

The effect of light on retinal cell viability, survival, or neurodegeneration of a retinal neuronal cell in the cell culture may be determined according to methods described herein and practiced in the art. The retinal cell culture light stress model described herein may be used as model for diseases that affect photoreceptor cells, for example, macular degeneration. In one embodiment of the invention, the retinal cell culture is exposed to light, particularly blue light, which decreases the survival or kills photoreceptor cells without killing any of the other major retinal cell types that are present in the cell culture system described herein. By way of example, the retinal cell culture system prepared as described herein, when exposed to 6000 lux of white light for 48 hours results in death of photoreceptor cells (over 95%); however, survival of ganglion cells was not reduced or adversely affected.

This model may be also used for studying cellular processes that underlie the pathology of a neurodegenerative diseases or disorders, particularly retinal diseases and disorders. By way of example, light stress affects retinal cells by inducing inappropriate activation of apoptosis (programmed cell death), which can contribute to a variety of pathological disease states. Apoptosis can be determined by a variety of methods known in the art and disclosed herein.

The light stress model may also be useful in a method for identifying agents or articles (e.g., a filter, lens, or other physical article) that block light from harming the eye. As described in more detail herein, the model may be used in methods for identifying a bioactive agent that blocks, inhibits, or prevents light from decreasing survival of retinal cells (e.g., photoreceptor cells) or that decreases the progression of or reverses neurodegeneration. The agent thus acts like a filter at the cellular level to block out harmful light such as ultraviolet or blue light. By way of example, light output applied only above a retinal cell culture and measured below cells that were maintained in culture media containing phenol red (which acts as an acid-base indicator and tints the media red) was 25% less luminous (decreased intensity) than the level of light output measured above the cells. Thus, the red media had a filtering effect that protected photoreceptor cells from the light stress.

Cigarette Smoke Condensate as a Cell Stressor

In one embodiment, the retinal cell stressor is tobacco smoke, one or more compounds found in tobacco smoke, or cigarette smoke condensate. Smoking is believed to be a risk factor for developing macular degeneration (Delcourt et al., Arch. Ophthalmol. 116:1031-35 (1998)). Tobacco smoke contains numerous mutagenic and carcinogenic compounds such as polyaromatic hydrocarbons (PAHs), tobacco-specific nitrosamines (TSNAs), carbazole, phenol, and catechol. PAHs are a group of chemicals in which constituent atoms of carbon and hydrogen are linked by chemical bonds that form two or more rings. Thus PAHs are sometimes called polycyclic hydrocarbons or polynuclear aromatics. Examples of such chemical arrangements are anthracene (3 rings), pyrene (4 rings), benzo(a)pyrene (5 rings), and similar polycyclic compounds. Exposure of bovine retinal pigment epithelial cells to benzo(a)pyrene appeared to inhibit growth and replication of the cells (Patton et al., Exp. Eye Res. 74:513-522 (2002)).

Tobacco specific nitrosamines (TSNAs) are electrophilic alkylating agents that are potent carcinogens. TSNAs are formed by reactions involving free nitrate during processing and storage of tobacco and by combustion of tobacco that contains the alkaloids, nicotine and nomicotine, in a nitrate rich environment. Fresh-cut, green tobacco contains virtually no tobacco specific nitrosamines (see, e.g., U.S. Pat. Nos. 6,202,649 and 6,135,121). In contrast, cured tobacco products obtained according to conventional methods contain a number of nitrosamines, including N′-nitrosonomicotine (NNN) and 4-(N-nitrosomethylamino)-1-(3-pyridyl)-1-butanone (NNK).

Additional toxic compounds produced in cigarette smoke include carbazole, phenol, and catechol. Carbazole is a heterocyclic aromatic compound containing a dibenzopyrrole system and is a suspected carcinogen. The phenolic compounds present in cigarette smoke occur as a result of pyrolysis of the polyphenols chlorogenic acid and rutin. Phenolic compounds in tobacco smoke include catechol, phenol, hydroquinone, resorcinol, o-cresol, m-cresol, and p-cresol. Catechol is the most abundant phenol in tobacco smoke (80-400 μg/cigarette) and has been identified as a co-carcinogen with benzo[a]pyrene.

Cigarette smoke condensate (CSC) may be prepared according to methods described herein and known in the art or may be purchased from a vendor such as Murty Pharmaceuticals (Lexington, Ky.). A mechanical device such as an FTC Smoke Machine or Phipps-Bird 20-channel smoking machine may be used for generating tobacco smoke. Examples of cigarettes used for preparing CSC include 1R4F or 1R3F research cigarettes or the like (see, e.g., Meckley et al., Food Chem. Toxicol. 42:851-63 (2004); Putnam et al., Toxicol. In Vitro 16:599-607 (2002)). To prepare CSC, for example, particulate constituents of tobacco smoke that is generated by one or more cigarettes may be deposited or collected on a filter, such as a glass fiber filter or another filter that is inert during the extraction process. Compounds are extracted from the filters using a solvent, for example, dimethyl sulfoxide (DMSO). The extraction procedure may also include a mechanical force such as sonication that is useful for aiding the removal of the particulate matter from the filters.

The effect of tobacco smoke on survival of retinal cells, particularly retinal neuronal cells, or on neurodegeneration of the retinal neuronal cells may be determined using the retinal cell culture system described herein. A retinal cell culture may be exposed to cigarette smoke condensate, tobacco smoke, or to one or more constituent compounds of tobacco smoke, including but not limited to the compounds discussed herein. The retinal cells may be exposed to a CSC cell stressor prior to culture of the retinal cells or for a period of time during the culture of the cells. Cells may be exposed to CSC for at least about 3 hours, 6 hours, 9 hours, 12, hours, 18 hours, 24 hours, or 2 days, 3 days, 4 days, 5 days, 6 days, or a week, 2 weeks, 4 weeks, 2 months, 4 months, or longer, or for any period of time between the time periods enumerated. The effect of the cell stressor on cell viability, survival, or alternatively on neurodegeneration, of the retinal cells in the cell culture may be determined according to methods described herein and known in the art.

Cigarette Smoke Condensate Plus Light as a Stressor

A retinal neuronal cell culture may be exposed to more than one cell stressor, for example, the culture may be exposed to at least two retinal cell stressors. For example, one retinal cell stressor may be cigarette smoke condensate and a second cell stressor may be light as described herein.

A retinal neuronal cell culture as described herein may be exposed to two cell stressors such as cigarette smoke condensate and a light source, separately or together, and then cultured. Alternatively, the retinal cell culture may be exposed to two cell stressors such as cigarette smoke condensate and a light source, separately or together, during the culture of the retinal neuronal cells. In certain embodiments, the retinal neuronal cells may be exposed to either one or both of the cell stressors prior to culturing the cells, or the cells may be exposed to one cell stressor prior to culture and then exposed to either one or both of the cell stressors during culture of the cells. The effect of the cell stressors on survival, or alternatively neurodegeneration, of the retinal cells in the cell culture may be determined according to methods described herein and known in the art. The time of exposure of the retinal neuronal cell culture to each cell stressor may differ. Cells may be exposed to CSC and/or light for at least about 3 hours, 6 hours, 9 hours, 12, hours, 18 hours, 24 hours, or 2 days, 3 days, 4 days, 5 days, 6 days, or a week, at least two weeks, and at least one month, or longer, or for any period of time between the time periods enumerated.

As described herein for culture of retinal cells exposed to a light stressor, the light may be emitted from at least one fluorescent light, incandescent light, or at least one light-emitting diode. The exposure may be intermittent or constant, and the duration of exposure may be varied. Alternatively, light stress may be applied as a light shock whereby cells at some point prior to or during cell culture may be protected from exposure to any light source and then exposed to a light stress. The light source may be inserted into a tissue culture incubator to provide continuous exposure or to regulate exposure during the time that the retinal cells are cultured.

The effect of the cell stressors on survival, or alternatively neurodegeneration, of the retinal cells in the cell culture may be determined according to methods described herein and known in the art. The retinal cell culture system described herein may be used as model for diseases that affect photoreceptor cells, for example, macular degeneration. When a light stressor is combined with a CSC stressor, the number of photoreceptor cells that survive is reduced compared to the number of photoreceptor cells that survive when exposed to CSC alone.

The retinal cell culture system comprising a CSC stressor and a light stressor may be also used for studying cellular processes that underlie the pathology of a neurodegenerative disease or disorder, particularly a retinal disease or disorder. For instance, such stressors may affect retinal cells by inducing inappropriate activation of apoptosis (programmed cell death), which can contribute to a variety of pathological disease states. Apoptosis can be determined by a variety of methods known in the art and described herein.

Physical Stressor: Increased Hydrostatic Pressure

In one embodiment of the invention, the retinal cell stressor is a physical cell stressor such as elevated hydrostatic pressure (pressure exerted by a liquid, which may be applied by methods described herein and practiced in the art such as, for example, increasing atmospheric pressure). Elevated intraocular pressure (IOP) is known in the art to correlate with glaucoma in patients. Ocular cells exposed to a hydrostatic pressure of 50 mm mercury (Hg) did not appear to have decreased viability, but morphological changes were observed as well as changes in distribution of actin stress fibers in certain cells (see Wax et al., Br. J. Ophthalmol. 84:423-28 (2000)). In one embodiment, the retinal cell culture system comprises isolated mature retinal cells, including retinal neuronal cells, and increased or elevated hydrostatic pressure (or atmospheric pressure) as a cell stressor. Cells may be exposed to a pressure that is 40, 45, 50, 55, 60, 70, 75, 80, 100, 110, 120, or 130 mm Hg (or at any pressure between the mm Hg enumerated). Increased pressure may be applied using methods described herein and known to a skilled artisan, for example, by using a pressure incubator (see, e.g., Healey et al., J. Vasc. Surg. 38:1099-105 (2003)) or by placing a pressure chamber within a tissue culture incubator (see, e.g., Wax et al., supra; see also Vouyouka et al., J. Surg. Res. 110:344-51 (2003)). The retinal neuronal cell culture system may be exposed to increased atmospheric pressure for at least 6 hours, 9 hours, 12, hours, 18 hours, 24 hours, or 2 days, 3 days, 4 days, 5 days, 6 days, or a week, at least two weeks, and at least one month (4 weeks), or longer, or for any period of time between the time periods enumerated.

One or more culture conditions may be adjusted so that the effect of the physical stressor, such as increased hydrostatic pressure, on the retinal cells can be more readily observed. For example, the concentration or percent of fetal bovine serum may be reduced or eliminated from the cell culture when cells are exposed to increased pressure.

In another embodiment, the retinal cell culture system comprises increased hydrostatic pressure (or increased atmospheric pressure) as one cell stressor and a second cell stressor. The retinal neuronal cells may be exposed to increased pressure concomitantly with the second stressor or the cells may be exposed first to one cell stressor and then to the second stressor. In alternative embodiments, the retinal neuronal cells may be exposed to either one or both of the cell stressors prior to culturing the cells; alternatively, the cells may be exposed to one cell stressor prior to culture and then exposed to either one or both of the cell stressors during culture of the cells. The effect of the cell stressors on retinal cell viability, survival, or neurodegeneration of a retinal neuronal cell, may be determined according to methods described herein and known in the art.

### Chemical Stressors: Retinoid N-retinylidene-N-retinyl-ethanolamine (A2E) Cell Stressor

In another embodiment, the stressor is a chemical. For example, the chemical stressor is a vitamin A derivative, such as retinoid N-retinylidene-N-retinyl-ethanolamine (A2E), or a derivative of A2E. A2E stress may include any one or more of A2E isomers including, such as iso-A2E (13-Z photo-isomer of A2E (see, e.g., Parish et al., Proc. Natl. Acad. Sci. USA 95:14609-13 (1998); Ben-Shabat et al., Angew. Chem. Int. Ed. 41:814-17 (2002)), or the stress may include all isoforms of A2E. A2E is a component of retinal lipfuscin, which according to non-limiting theory is formed from retinal, digested rhodopsin, and ethanolamine (a cell membrane component), in retinal pigment epithelial cells that line the photoreceptor rods and cones during processing of cellular debris (see, e.g., Parish et al., supra; Mata et al., Proc. Natl. Acad. Sci. USA 97:7154-59 (2000)). Accumulation of A2E has been hypothesized to contribute to development of age-related neurodegeneration of retinal cells, particularly macular degeneration. Exposure of the retinal neuronal cell culture system described herein to A2E results in selective killing of certain cells, particularly photoreceptor cells, that are present in the retinal cell culture system.

The photoreceptors in the retina, designed to initiate the cascade of events that link the incoming light to the sensation of “vision,” are susceptible to damage by light, particularly blue light. The damage can lead to cell death and diseases, particularly the dry form of macular degeneration. The turnover of retinal, an essential element of the visual process, is the basis of the events that lead to damage. Free retinal, absorbing in the blue region of the visible spectrum, is phototoxic and is a precursor of the (photo)toxic compound A2E, which specifically targets cytochrome oxidase and thereby induces cell death by apoptosis.

In one embodiment, the retinal cell culture system may be exposed to A2E at any concentration between 1 pM and 200 μM (e.g., 1 pM, 10 pM, 100 pM, 250 pM, 500 pM, 750 pM, 1 nM, 10 nM, 50 nM, 100 nM, 250 nM, 500 nM, 750 nM, 1 μM, 2 μM, 5 μM, 7.5 μM, 10 μM, 15 μM, 20 μM, 25 μM, 40 μM, 50 μM, 75 μM, 100 μM, 120 ∥M, 200 μM); or 250 μM, 500 μM, or 750 μM), between 1 μM and 40 μM, or between 10 μM and 20 μM, for a period of time, for example, between 2 and 48 hours or between 12 and 36 hours. In another embodiment, the cell culture may be exposed to lower concentrations of A2E (for example, between 1 pM and 10 μM or between 1 nM and 1 μM) for longer times (such as about one week, about two weeks, or about one month (4 weeks)). By way of example, the retinal cell culture system prepared as described herein when exposed to 20 μM A2E for 48 hours results in death of photoreceptor cells (more than 90% of photoreceptor cells die compared to photoreceptor cells not exposed to A2E); survival of ganglion cells is not adversely affected (i.e., ganglion cell viability is not reduced).

In certain other embodiments, more than one stressor may be applied to the retinal cell culture system. For example, a culture may be exposed to a light stressor and a chemical stressor such as A2E according to methods and techniques described herein. Additional stressors that are known in the art and described herein, including but not limited to glucose oxygen deprivation, pressure, and neurotoxins, may be combined with either a light stressor or a chemical stressor or both stressors.

Chemical Cell Stressor: Glutamate

In another embodiment, a retinal cell culture system includes glutamate as a cell stressor. In the mammalian central nervous system (CNS), the transmission of nerve impulses is controlled by the interaction between a neurotransmitter, which is released by a sending neuron, and a surface receptor on a receiving neuron, which causes excitation of this receiving neuron. Excitatory amino acids (EAAs), principally glutamic acid (the primary excitatory neurotransmitter) and aspartic acid, mediate the major excitatory pathway in the mammalian central nervous system. Thus, glutamic acid can bring about changes in the postsynaptic neuron that reflect the strength of the incoming neural signals. The receptors that respond to glutamate are called excitatory amino acid receptors (EAA receptors) (see, e.g., Watkins et al., Trans. Pharm. Sci. 11:25 (1990); Monaghan et al., Annu. Rev. Pharmacol. Toxicol. 29:365 (1989); Watkins et al., Annu. Rev. Pharmacol. Toxicol. 21:165 (1981)). The excitatory amino acids play a role in a variety of physiological processes, such as long-term potentiation (learning and memory), the development of synaptic plasticity, motor control, respiration, cardiovascular regulation, and sensory perception.

Excitatory amino acid receptors are classified into two general types: ionotropic and metabotropic. The ionotropic receptors contain ligand-gated ion channels and mediate ion fluxes for signaling, while the metabotropic receptors use G-proteins for signaling. Both types of receptors appear not only to mediate normal synaptic transmission along excitatory pathways, but also to participate in the modification of synaptic connections during development and throughout life (see, e.g., Schoepp et al., Trends in Pharmacol. Sci. 11:508 (1990); McDonald et al., Brain Res. Rev. 15:41 (1990)).

Further sub-classification of the ionotropic EAA glutamate receptors is based upon the agonists (stimulating agents) other than glutamic and aspartic acid that selectively activate the receptors. The at least three subtypes of the ionotropic receptors are defined by the depolarizing actions of allosteric modulators: a receptor responsive to N-methyl-D-aspartate (NMDA); a receptor responsive to alpha-amino-3-hydroxy-5-methylisoxazole-4-propionic acid (AMPA); and a receptor responsive to kainic acid (KA). The NMDA receptor controls the flow of both divalent (Ca⁺⁺) and monovalent (Na⁺, K⁺) ions into the postsynaptic neural cell. The AMPA and KA receptors also regulate the flow into postsynaptic cells of monovalent K⁺ and Na⁺, and occasionally divalent calcium (Ca⁺⁺). Other glutamate agonists in addition to NMDA, AMPA, and KA include aspartate, ACPD, quisqualic acid, ibotenic acid, and quinolinic acid. A glutamate agonist may be included as a retinal cell stressor in the mature retinal cell culture system at concentrations and for a duration and at times as described herein for the inclusion of glutamate as a cell stressor.

The G-protein excitatory amino acid receptor is coupled to multiple second messenger systems that lead to enhanced phosphoinositide hydrolysis, activation of phospholipase D, increased or decreased c-AMP formation, and/or changes in ion channel function (see, e.g., Schoepp et al., Trends in Pharmacol. Sci. 14:13 (1993)). The metabotropic EAA receptors are divided into three sub-groups, which are unrelated to ionotropic receptors, and are coupled via G-proteins to intracellular second messengers. These metabotropic EAA receptors are classified based on receptor homology and second messenger linkages. EAA receptors have been implicated during development in specifying neuronal architecture and synaptic connectivity and may be involved in experience-dependent synaptic modifications.

These receptors appear to be involved in a broad spectrum of CNS disorders. For example, during brain ischemia caused by stroke or traumatic injury, excessive amounts of the EAA glutamic acid are released from damaged or oxygen-deprived neurons. Binding of this excess glutamic acid to the postsynaptic glutamate receptors opens their ligand-gated ion channels, thereby allowing an ion influx that in turn activates a biochemical cascade resulting in protein, nucleic acid, and lipid degradation, and cell death. This phenomenon, known as excitotoxicity, may also be responsible for the neurological damage associated with other disorders ranging from hypoglycemia, ischemia, and epilepsy to chronic neurodegeneration that occurs in Huntington's, Parkinson's, and Alzheimer's diseases (see, e.g., Kannurpatti et al., Neurochem. Int. 44:361-69 (2004); Curr. Top. Med. Chem. 4:149-77 (2004); Swanson et al., Curr. Mol. Med. 4:193-205 (2004)). Excessive activation of ionotropic receptors and group I metabotropic receptors may result in neuronal death. Many neurodegenerative conditions, including Parkinson's disease, Alzheimer's disease, cerebral ischaemia, epilepsy, Huntington's chorea and amyotrophic lateral sclerosis (ALS), have been linked to disturbed glutamate homeostasis (Tortarolo et al., J. Neurochem. 88:481-93 (2004); Lipton et al, New Eng. J. Med. 330:613-22 (1994); Gegelashvili et al., Mol. Pharmacol. 52:6-15 (1997); Robinson et al., Adv. Pharmacol. 37:69-115 (1997)).

In glaucoma, the increased release of glutamate is a major cause of retinal ganglion cell death (see, e.g., El-Remessy et al., Am. J. Pathol. 163:1997-2008 (2003)). Extracellular glutamate concentrations are maintained within physiological levels exclusively by glutamate transporters, permitting normal excitatory transmission as well as protecting against excitotoxicity (Robinson et al., Adv Pharmacol. 37:69-115 (1997)). Nerve damage may be caused by abnormal accumulation of glutamate that leads to overexcitation of the receiving nerve cell or may be caused by oversensitive glutamate receptors on the receiving nerve cell.

The cell culture system described herein comprising mature retinal cells including retinal neuronal cells may comprise glutamate or a derivative thereof (see, e.g., U.S. Patent Application No. 2002/0115688) or a glutamate agonist as a cell stressor (see Luo et al., supra). The concentration of glutamate added to a retinal cell culture may be between 0.5 nM-100 μM, such as about 0.5 nM, 1 nM, 2 nM, 4 nM, 5 nM, 7.5 nM, 10 nM, 20 nM, 40 nM, 50 nM, 75 nM, 100 nM, 0.1 μM, 0.5 μM, 1 μM, 2 μM, 4 μM, 5 μM, 7.5 μM, 10 μM, 20 μM, 25 μM, 40 μM, 50 μM, 60 μM, 75 μM, or 100 μM, or between 100 μM and 1 mM, such as about 150 μM, 200 μM, 250 μM, 300 μM, 400 μM, 500 μM, 600 μM, 750 μM, 800 μM, 900 μM, and 1000 μM (1 mM). Glutamate acting as a cell stressor may be added to a retinal cell culture at the time the freshly harvested (isolated) retinal cells are prepared and plated for tissue culture. Alternatively, glutamate may be added at a time subsequent to plating and establishment of the retinal cells in culture. Glutamate may be added one day after plating the retinal cells, two days, three days, four days, five days, six days, or 7 days (one week), 2 weeks, 3 weeks, 4 weeks, or 6 weeks, or longer, after plating of the cells.

Glutamate may also be combined with one or more other cell stressors described herein, for example, light stress, CSC, A2E stress, or increased hydrostatic pressure. As described herein when a retinal cell culture is exposed to two or more cell stressors, glutamate and one or more other cell stressors may be applied or added to the cell culture together at the same time or may be applied or added to the cell culture separately at different times and in any order. The time of exposure to each cell stressor may be different or may be the same.

A glutamate stress retinal cell culture model with or without additional cell stressors may be used for identifying bioactive agents that alter (i.e., increase or decrease in a statistically significant manner) retinal cell, including retinal neuronal cell, viability, survival, or neurodegeneration according to methods described herein. A bioactive agent that enhances (extends or promotes) survival of retinal neurons or inhibits or impairs (slows the progression of) neurodegeneration may affect any one of a number of different pathways and receptors that are affected by excitotoxic mechanisms. For example, activation of glutamate receptors can trigger death of neurons and some types of glial cells, particularly when cells are also subjected to adverse conditions such as reduced levels of oxygen or glucose, increased levels of oxidative stress, exposure to toxins, or a genetic mutation. Excitotoxic death that occurs as a result of one or more of these adverse conditions may involve excessive calcium influx, release of calcium from internal cell organelles, radical oxygen species production, and engagement of apoptotic cascades. See, e.g., Mattson, Neuromolecular Med. 3:65-94 (2003); Atlante et al., FEBS Lett. 497:1-5 (2001). A bioactive agent identified in screening assays described herein in which glutamate is a cell stressor may be useful for reducing excitotoxic cell death by interacting with one or more components of one or more of these pathways.

Screening Neurological Targets for Drug Discovery

Neurodegenerative diseases are a major source of morbidity. An in vitro cell culture model comprising neuronal cells would be of benefit to drug discovery for identifying agents for treating neurodegenerative diseases and disorders. Because culturing of post-mitotic neuronal cells has been difficult, a good paradigm is critical when screening drugs relevant to neurologic and ophthalmic diseases. The response of target molecules to potential drug candidates is likely to depend at least in part on the cellular environment of the target molecule. Thus, using cultured cells that are closely related to the cell types that are ultimately to be treated with the drug is an important consideration for developing and using screening assays.

Proper validation of drug/therapeutic candidate agents entails identification and evaluation of tissue-specific cultured cells for use within a cell-based screening system. In the field of neurobiology, cell lines such as PC12 cells (derived from a rat pheochromocytoma), NT2 cells (derived from a human teratocarcinoma), or human neuroblastoma cell lines have been used to screen drug candidates. While these cells have some characteristics of prototypic neurons, these cells are tumor-derived. The cell lines are therefore considered to be different from physiologically normal neuronal cells in that the cells of the tumor-derived cell lines may not form a site-appropriate mix of neuronal and non-neuronal cells representative of the mixture and relationship of cells found in intact animals. In addition, such cells commonly have abnormal karyotypes with extra copies of chromosomes or genes, the expression of which could ultimately affect the action of many drugs in a way that might not be observed in non-tumor derived neuronal cells.

In one embodiment, the in vitro retinal cell culture stress model described herein is used for identification and biological testing of bioactive agents and compounds, particularly neuroactive agents and compounds, or materials that may be suitable for treatment of neurological diseases or disorders in general, and for treatment of degenerative diseases of the eye and brain in particular. In another embodiment, screening methods may comprise the in vitro retinal cell culture system in the absence of a cell stressor to identify a cell stressor or to identify a bioactive agent that may be suitable for treating a subject who has a neurological, particularly, a retinal disease or disorder. Methods for identifying a bioactive agent that alters (increases or decreases in a statistically significant manner) viability of a mature retinal cell comprise contacting (combining, mixing, or otherwise permitting interaction of) a candidate agent with the mature retinal cells present in a retinal cell culture system (in the absence or presence of one or more cell stressors) under conditions and for a time sufficient to permit interaction between the candidate agent and the cell culture system, and then comparing the viability of a plurality of mature retinal cells in the presence of the candidate agent with the viability of a plurality of mature retinal cells in the absence of the candidate agent. The plurality of retinal cells that are not exposed to a candidate agent may be prepared simultaneously from the same retinal tissue as the retinal cells that are exposed to a candidate agent. Alternatively, the viability of retinal cells in the presence of an agent may be quantified and compared to viability of a standard retinal cell culture (i.e., a retinal cell culture system as described herein that provides repeatedly consistent, reliable, and precise determinations of retinal cell viability).

Through use of the methods described herein, agents may be selected and tested that are useful for treating diseases and disorders of the central nervous system and retina, including but not limited to neurodegenerative diseases, epilepsy, glaucoma, macular degeneration, diabetic retinopathy, retinal detachment, retinal blood vessel (artery or vein) occlusion, retinitis pigmentosa, inflammatory retinal diseases, optic neuropathy, and retinal disorders associated with other degenerative diseases such as Alzheimer's disease, Parkinson's disease, or multiple sclerosis, or associated with AIDS. The cultured mature neurons provided herein are particularly useful for screening candidate bioactive agents to identify a bioactive agent that may enable or effect regeneration of CNS tissue that has been damaged by disease. For example, the presence of photoreceptors with an intact outer segment is relevant in such an assay to identify compounds useful for treating neurodegenerative eye diseases.

In one embodiment, one or more candidate bioactive agents are incorporated into screening assays comprising the retinal cell culture stress model system described herein to determine whether the bioactive agent increases viability (i.e., increases in a statistically significant or biologically significant manner) of a plurality of retinal cells. A person skilled in the art would readily appreciate and understand that as described herein a retinal cell which exhibits increased viability means that the length of time that a retinal cell survives in the cell culture system is increased (increased lifespan) and/or that the retinal cell maintains a biological or biochemical function (normal metabolism and organelle function; lack of apoptosis; etc.) compared with a retinal cell cultured in an appropriate control cell system (e.g., the cell culture system described herein in the absence of the candidate agent). Increased viability of a retinal cell may be indicated by delayed cell death or a reduced number of dead or dying cells; maintenance of structure and/or morphology; lack of or delayed initiation of apoptosis; delay, inhibition, slowed progression, and/or abrogation of retinal neuronal cell neurodegeneration or delaying or abrogating or preventing the effects of neuronal cell injury. Methods and techniques for determining viability of a retinal cell and thus whether a retinal cell exhibits increased viability are described in greater detail herein and are known to persons skilled in the art (see also, e.g., methods and techniques described for identifying a retinal cell stressor).

In one embodiment, one or more candidate bioactive agents are incorporated into screening assays comprising the retinal cell culture stress model system to determine whether the bioactive agent is capable of altering neurodegeneration of neuronal cells (impairing, inhibiting, preventing, abrogating, reducing, slowing the progression of, or accelerating in a statistically significant manner). A preferred bioactive agent is one that inhibits, reduces, abrogates, slows the progression of, or impairs neurodegeneration of a neuronal cell, particularly a retinal neuronal cell, that is capable of regenerating a neuronal cell, and/or that is capable of enhancing or prolonging survival (promoting, improving, or increasing survival, thus delaying injury and/or death) of a neuronal cell. A bioactive agent that inhibits neurodegeneration of a neuronal cell may be identified by contacting (mixing, combining, or otherwise permitting interaction between the agent and retinal cells of the cell culture system), for example, a candidate agent from a library of agents as described herein, with the cell culture system under conditions and for a time sufficient to permit interaction between a candidate agent and the retinal cells, particularly the mature retinal neuronal cells of the cell culture system described herein.

A bioactive agent may act directly upon a retinal neuronal cell in a manner that affects survival or neurodegeneration (or neuronal cell injury) of the cell. Alternatively, a bioactive agent may act indirectly by interacting with one retinal cell type that consequently, via a biological response to the agent, affects viability, that is survival and/or neurodegeneration, of another retinal cell. Not wishing to be bound by theory, glial cells such as Müller glial cells that are associated with retinal neurons and interact with retinal neurons such that the Müller glial cells support the metabolic function of the neurons, may be acted upon by a bioactive agent. The effect of the agent on the biological or biochemical function of a Müller glial cell may in turn affect the metabolism, viability, and survival of the associated retinal neuron(s). For instance, viability, survival, or neurodegeneration of a retinal neuronal cell may be indirectly affected or altered in a biologically significant manner by a candidate agent that maintains viability or enhances survival of a Müller glial cell.

In certain embodiments, the methods described herein may be used for identifying a bioactive agent that alters viability (i.e., alters survival and/or neurodegeneration and/or neuronal cell injury) of one, two, three, or more, or all retinal cell types and may also be used to identify an agent that alters viability of one, two, three, or more, or all retinal neuronal cell types (amacrine cell, a photoreceptor cell, a ganglion cell, horizontal cell, and bipolar cell). In certain other embodiments, the screening methods may be used to identify a bioactive agent that alters viability (preferably enhances survival and/or inhibits neurodegeneration or cell injury) of one retinal neuronal cell type, such as an amacrine cell, a photoreceptor cell, or a ganglion cell, horizontal cell, or bipolar cell.

In one embodiment, a method for identifying a bioactive agent that alters viability of a retinal cell includes light as a cell stressor. In another embodiment, A2E is added as a cell stressor. A method for identifying a bioactive agent may include more than one cell stressor. For example, the light plus cigarette smoke condensate stress model is used to identify a bioactive agent that impairs or inhibits the activity of A2E such that A2E is inhibited or blocked from acting as a stressor in the retinal cell culture system. As described herein, A2E is a component of retinal lipfuscin, which according to non-limiting theory is formed from retinal, digested rhodopsin, and ethanolamine (a cell membrane component), in retinal pigment epithelial cells that line the photoreceptor rods and cones during processing of cellular debris (see, e.g., Parish et al., supra; Mata et al., Proc. Natl. Acad. Sci. USA 97:7154-59 (2000)). Accumulation of A2E may play some role in development of age-related neurodegeneration of retinal cells, particularly macular degeneration. Exposure of the retinal cell culture system described herein to A2E results in selective killing of certain cells, particularly photoreceptor cells, that are present in the retinal cell culture.

A bioactive agent may include, for example, a peptide, a polypeptide (for example, a ligand that binds to a retinal cell receptor, such as a retinal neuronal cell receptor, a growth factor, trophic factor, or the like), an oligonucleotide or polynucleotide, antibody or binding fragment thereof, lipid, hormone, or small molecule. Candidate agents for use in a method of screening for a bioactive agent that is capable of altering (increasing or decreasing in a statistically significant manner) neurodegeneration of neuronal cells or survival of cells, such as retinal neuronal cells, may be provided as “libraries” or collections of compounds, compositions, or molecules. Such molecules typically include compounds known in the art as “small molecules” that have molecular weights less than 10⁵ daltons, less than 10⁴ daltons, or less than 10³ daltons. Candidate agents further may be provided as members of a combinatorial library, which includes synthetic agents prepared according to a plurality of predetermined chemical reactions performed in a plurality of reaction vessels. The resulting products comprise a library that can be screened and then followed by iterative selection and synthesis procedures to provide, for example, a synthetic combinatorial library of peptides (see, e.g., PCT/US91/08694, PCT/US91/04666) or other compositions that may include small molecules as provided herein (see, e.g., PCT/US94/08542, U.S. Pat. No. 5,798,035, U.S. Pat. No. 5,789,172, U.S. Pat. No. 5,751,629). Those having ordinary skill in the art will appreciate that a diverse assortment of such libraries may be prepared by a skilled artisan according to established procedures. Bioactive agents that are believed to or known to interact with neurons or retinal cells (including retinal neurons) or to affect neurological activity (that is alter the structure and/or function of a neuron) may be included in the methods described herein to identify an agent that alters viability of a retinal cell in particular.

Preferably, a bioactive agent enhances survival of neuronal cells such as retinal neuronal cells, that is, the agent promotes survival or prolongs survival such that the time period in which neuronal cells are viable is extended. The ability of a candidate agent to enhance cell survival or impair, inhibit, or impede neurodegeneration may be determined by any one of several methods described herein and practiced by those skilled in the art. For example, changes in cell morphology in the absence and presence of a candidate agent may be determined by visual inspection such as by light microscopy, confocal microscopy, or other microscopy methods known in the art. Survival of cells can be determined by counting viable and/or nonviable cells, for instance. Immunochemical or immunohistological techniques (such as fixed cell staining or flow cytometry) may be used to identify and evaluate cytoskeletal structure (e.g., by using antibodies specific for cytoskeletal proteins such as glial fibrillary acidic protein, fibronectin, actin, vimentin, tubulin, or the like) or to evaluate expression of cell markers as described herein. The effect of a candidate agent on cell integrity, morphology, maturation, and/or survival may also be determined by measuring the phosphorylation state of neuronal cell polypeptides, for example, cytoskeletal polypeptides (see, e.g., Sharma et al., J. Biol. Chem. 274:9600-06 (1999); Li et al., J. Neurosci. 20:6055-62 (2000)).

Enhanced survival (or prolonged or extended survival) of one or more retinal cell types in the presence of a bioactive agent indicates that the bioactive agent may be an effective agent for treatment of a neurodegenerative disease, particularly a retinal disease or disorder. Cell survival and enhanced cell survival may be determined according to methods described herein and known to a skilled artisan including viability assays and assays for detecting expression of retinal cell marker proteins. For determining enhanced survival of photoreceptor cells, opsins may be detected, for instance, including the protein rhodopsin that is expressed by rods. Rhodopsin, which is composed of the protein opsin and retinal (a vitamin A form), is located in the membrane of the photoreceptor cell in the retina of the eye and catalyzes the only light sensitive step in vision. The 11-cis-retinal chromophore lies in a pocket of the protein and is isomerized to all-trans retinal when light is absorbed. The isomerization of retinal leads to a change of the shape of rhodopsin, which triggers a cascade of reactions that lead to a nerve impulse that is transmitted to the brain by the optical nerve.

Viability (or survival) of one or more retinal cell types that are present in the cell culture system described herein may be determined according to methods described herein (see also, e.g., methods and techniques described for identifying a retinal cell stressor) and which are familiar to a skilled artisan. For example, viable cells may be differentiated from non-viable cells by uptake of particular dyes, such as trypan blue. Alternatively, cell death and cell lysis may be quantified by measuring cellular metabolites or enzymes, such as alkaline and acid phosphatase, glutamate-oxalacetate transaminase, glutamate pryuvate transaminase, argininosuccinate lyase, and lactate dehydrogenase, that are released into cell culture media supernatant from the damaged cells (e.g., via damaged or compromised plasma membranes) or upon cell expiration. For example, viability assays may be employed that use esterase substrates, stain nucleic acids, or that measure oxidation or reduction (see Molecular Probes, Eugene, Oreg., Invitrogen Life Sciences, Carlsbad, Calif.). Viability of living cells that are not actively dividing, such as retinal neuronal cells, may be determined by evaluating one or more metabolic processes. Such methods incorporate reagents that may be detected by calorimetric or fluorimetric analyses. Companies that provide assay kits for determining cell viability/vitality or cytotoxicity include Roche Applied Science, Indianapolis, Ind. and Molecular Probes.

Viability of one or more retinal cell types in the cell culture system may be determined by assessing survival of the one, two, three, or more retinal cell types. Viability or survival of retinal cells in the cell culture system in the absence or presence of one or more cell stressors may be determined, as well as viability (survival) in the absence or presence of a candidate bioactive agent. Preferably, a bioactive agent that is identified according to methods described herein enhances or prolongs survival of one or more retinal cell types. Survival may be determined by comparing the number (or percent) of retinal cells exposed to an agent that are viable over a defined period of time relative to the number (or percent) of retinal cells not exposed to an agent that are viable over the same defined time period. Survival of retinal cells in the cell culture system may be compared during the time the cells are exposed to a candidate bioactive agent or may be compared for a period(s) of time after the bioactive agent is removed from the cell culture system. The time period may be 1 day, 2-3 days, 4-7 days, 7-14 days, or 14-28 days, 2 months, 4, months, or longer.

A bioactive agent that effectively alters, preferably inhibits, impairs, slows the progression of, prevents, or decreases neurodegeneration or neuronal cell injury of a retinal neuronal cell may be identified by techniques known in the art and described herein for determining the effects of the agent on neuronal cell structure or morphology; expression of neuronal cell markers (e.g., β3-tubulin, rhodopsin, recoverin, visinin, calretinin, calbindin, neurofilament (NFM), Thy-1, tau, microtubule-associated protein 2, neuron-specific enolase, protein gene product 95, and the like (see, e.g., Espanel et al., Int. J. Dev. Biol. 41:469-76 (1997); Ehrlich et al., Exp. Neurol. 167:215-26 (2001); Kosik et al., J. Neurosci. 7:3142-53 (1987); Luo et al., supra)); and/or cell survival (i.e., cell viability or length of time until cell death). Antibodies that may be used include antibodies that specifically bind to a protein that is expressed by specific cell types (e.g., opsins expressed by photoreceptor cells, for example, rhodopsin expressed by rods; β3-tubulin expressed by interneurons and ganglion cells; and NFM expressed by ganglion cells), and include antibodies that specifically identify a cell marker expressed by a retinal cell from a specific animal source.

A bioactive agent identified using the cell culture and assay methods described herein may affect regeneration of retinal neuronal cells. Regeneration of neuronal cells or proliferation of neuronal cells may be determined by any of several methods known in the art, for example, by measuring incorporation of labeled deoxyribonucleotides or ribonucleotides or derivatives thereof, such as tritiated thymidine, or such as by measuring incorporation of bromodeoxyuridine (BrdU), which can be detected by using antibodies that specifically bind to BrdU.

Viability, cell survival or, alternatively cell death, may also be determined according to methods described herein and known in the art for determining whether cells are apoptotic (for example, annexin V binding, DNA fragmentation assays (such as terminal deoxynucleotide transferase-mediated dUTP nick-end labeling (TUNEL)); caspase activation; mitochondrial membrane potential breakdown; marker analysis, e.g., poly(ADP-ribose) polymerase (PARP); detection with antibodies specific for enzymes or polypeptides expressed during apoptosis (e.g., an anti-caspase-3 antibody; etc.).

In some instances, such methods may enable identification of candidate bioactive or therapeutic agents that not only improve the symptoms directly or indirectly related to neurodegeneration that may be manifested by a subject or patient, but also act to reverse the state of neurodegeneration. The disclosed methods and cell culture model systems permit precise measurements of specific interactions occurring between neurons, as well as enabling detailed analysis of subtleties in neuron structure. For instance, the methods and cultured cells described herein are compatible with neurochips, cell-based biosensors, and other multielectrode or electrophysiologic devices for stimulating and recording data from cultured neurons (see, for instance, M. P. Maher et al., J. Neurosci. Meth. 87:45-56, 1999; K. H. Gilchrist et al., Biosensors & Bioelectronics 16:557-64, 2001).

Uses for the Retinal Cell Culture Stress Models

The in vitro retinal cell culture stress models described herein may be used for identifying bioactive molecules that enhance survival or prevent or inhibit cell death and/or degeneration of retinal cells. In addition, this model may be useful for investigating long-term effects of bioactive molecules that may not exhibit their effects during short time frames. Further, this system may find use in detecting and/or identifying various toxins or neurotoxins. A bioactive molecule, toxin, or neurotoxin thus identified may potentially be used as a stressor alone or in combination with one or more other stressors described herein. The availability of a long-term cell culture system may be particularly beneficial in the field of neurotoxicology because some chemicals and active agents exhibit toxic effects in low doses, but only over extended periods of time.

The methods and model systems described herein may also be applied to mature neuronal cells obtained from genetically mutated animals. For instance, mature neurons may be obtained from an animal that expresses the retinal dystrophic (rd/rd) allele. A comparison of wild-type and mutant neuronal cells in extended cell culture conditions may aid in identification of bioactive molecules, or in identification of up- or down-regulated moieties within these cells upon exposure to stresses, or to added or subtracted compounds or nutrients. Other animal models that carry characterized alleles relating to brain, eye, or other CNS disorders or diseases may be amenable for use as a source of mature differentiated cells (including mature neuronal cells) within the methods and systems described herein.

In addition, with the advent of novel technologies such as genomics and proteomics, thousands of new, relatively uncharacterized genes and proteins have been identified. One of the bottlenecks of drug discovery and development is determining how to prioritize thousands or millions of small molecule and proteinaceous therapeutic agent candidates that are available for high-throughput screening. Most of these high-throughput assay systems are based on test molecule stimulation or inhibition of target cell enzymatic activity, or on binding of a test molecule to a target molecule or target cell. Because in vivo systems feature complex interactions between target molecules or target cells and surrounding molecules within the target molecule's cellular environment or the target cell's surrounding tissue environment, predicting the manner in which a candidate molecule identified by an isolated biochemical assay will affect the same target molecule or cell in an in vivo setting may be difficult. For example, certain target proteins, such as transcription factors and cell-surface receptors, often form multi-subunit complexes in order to exhibit biological function. Furthermore, the response of a target protein to a potential therapeutic agent is likely to be dependent on its cellular context. An assay using the retinal cell culture systems and methods described herein will represent the in vivo target molecule's cellular environment.

Another research and development bottleneck involves correlating genetic analysis or sequence information with functional biology in order to validate a therapeutic or diagnostic target. Bioinformatics and genomic technologies have identified new genes that map to regions of the chromosome associated with genetic mutations or defects that have been associated with biological diseases or disorders. However, identifying and analyzing the precise biological function of the thousands and millions of interesting genes (and their corresponding gene products) is proving to be extremely challenging. Without good cellular models, elucidating one or more biological functions of each protein within a cell is difficult. Thus, although bioinformatics and genomics techniques may identify potential disease-causing proteins and candidate therapeutic agents, characterizing the biological significance and function of each of such molecules continues to be difficult and time consuming. Consistent and reproducible cell-based assay systems and stress models, such as provided herein, will accelerate this functional analysis. Furthermore, use of the cultured neuronal cells as described herein may permit identification of bioactive agents that target intracellular functional units or other types of non-protein molecules, such as ribosomes, lipids, or carbohydrates.

The next generation of drug discovery platform technology may incorporate “cellomics.” Cellomics will use comprehensive analyses of in vitro or ex vivo cultured cells. Cell-based screening systems such as the retinal cell culture system described herein permits candidate biopharmaceutical agents to interact with corresponding target molecules in a more physiological state than in a simpler protein-target analysis.

The in vitro retinal cell stress model described herein may be used to study and elucidate underlying mechanisms of disease including common damage and recovery pathways that will enable the discovery and development of therapeutics for treating neurodegenerative diseases. Such an investigation of cellular properties can lead to development of improved disease models and improved disease modeling.

The stress model systems may also be used for detecting neuronal cell regeneration that occurs because of the potential presence of adult stem cells in the retinal cell culture. Müller glial cells, epithelial cells, neuronal cells, or other cell types may have the ability to serve as retinal stem cells and produce new neurons such as by means of transdifferentiation or reversion to a progenitor cell-like phenotype. Viral probes can be prepared that specifically detect dividing cells in this cell model. Newly formed cells can be detected by standard procedures such as immunoassays and other assays known in the art (for example, immunohistochemistry) to detect both a viral specific marker and a neuronal marker. Another method known in the art for detecting dividing cells is incorporation of bromodeoxyuridine (BrdU), which can be used in combination with an anti-BrdU specific antibody and an antibody specific for a neuron specific marker, to detect newly regenerated neurons in these stress model systems.

The stress model systems may be useful for determining the efficacy of potential therapeutics for neurodegenerative diseases. For example, recombinant polynucleotides and vectors can be added to the model system alone or in combination with reagents that may facilitate gene transfer. Transfection efficiency and/or the therapeutic effect of the polynucleotide can be monitored and assessed according to methods within the skill set of a person skilled in the art.

The methods and systems described herein may also be used to provide a source of neuronal cell RNA and DNA. For instance, the retinal neuronal cells cultured according to the described methods and systems may provide sufficient and appropriate material for construction of retinal neuronal cell cDNA libraries. In addition, such neuronal cell cultures may be useful in proteomics analyses as discussed herein.

The cell culture methods and systems described herein may be used for determining risk factors that increase the possibility that a subject will develop a retinal disease or disorder. In one embodiment, the cell culture system may be used to identify a retinal cell stressor (biological, chemical, or physical) that is present in the environment (inside or outside a structure or enclosed space), such as a pesticide, fungicide, herbicide, or other biocide, or a toxic building material, or other toxic chemical or material. The methods and systems described herein may also find use as a biosensor to detect molecules used for bioterrorism, and particularly as a biosensor to detect neurologically active molecules of bioterrorism. The disclosed methods and systems may also be used to identify and develop therapeutic agents that are capable of counteracting the effects of such molecules of bioterrorism.

Thus, the a retinal cell culture stress model comprises a cell culture system comprising mature (non-embryonic) retinal neuronal cells and other retinal cells that survive for extended periods of time in culture without inclusion of other types of non-retinal cells such as cells harvested from ciliary bodies within the eye or added purified or isolated glial cells or added stem cells. The retinal neuronal cells comprise all major retinal cell types (interneurons such as amacrine cells, horizontal cells, and bipolar cells; ganglion cells; and photoreceptor cells). The cell culture system provides extended survival of photoreceptor cells. Also provided are methods for screening bioactive molecules using the in vitro cell culture stress model systems (i.e., a cell culture system comprising mature retinal neuronal cells and at least one cell stressor).

Treatment of Neurodegenerative Diseases

In another embodiment, methods are provided for treating neurodegenerative diseases and disorders, particularly neurodegenerative retinal diseases as described herein. A subject in need of such treatment may be a human or non-human primate or other animal who has developed symptoms of a neurodegenerative retinal disease or who is at risk for developing a neurodegenerative disease. Treating such a subject (or patient) is understood to encompass preventing further cell death, or replacing, augmenting, repairing, or repopulating damaged tissue and cells by administering retinal neuronal cells. Such transplantation of retinal cells may be performed according to methods known in the art, and which include methods to minimize or prevent rejection of the transplanted cells by the host, which may include administering agents that suppress the host's immune response.

In one embodiment, retinal cells, including retinal neuronal cells, propagated in the extended retinal cell culture system described herein are administered to a subject (patient) in need thereof prior to the end-stage of a neurodegenerative disease, and preferably at a time point prior to initiation of neurodegeneration, or at a time point that will prevent, slow, or impair further neurodegeneration (that is, for example, soon after an initial diagnosis has been made). By way of example, a diagnosis of macular degeneration can be made at early stages of the disease. According to the present invention, introduction of retinal cells and more particularly photoreceptor cells at the time of diagnosis may delay, prevent, impair, or inhibit further neurodegeneration of photoreceptor cells.

The retinal cells may be introduced into a subject in need thereof according to standard transplantation procedures known in the medical arts, including grafting, near or at the site of dystrophic tissue, preferably into retinal tissue, and may also include injection of retinal neuronal cells into a site, for example, into the vitreous of the eye. The transplantation may be an autograft (neuronal cells from the subject to be treated); syngeneic graft (of the same strain, that is, having the same histocompatibility genes); allogeneic graft (same species, but different strains, that is, the donor and recipient have different histocompatibility genes); or xenogenic graft (donor and recipient belong to different species or genus). For transplantation in humans, non-human primates may be used as a source of retinal cells. Alternatively, transgenic animals, such as a transgenic pig, may be an acceptable source of retinal cells. Procedures and methods for increasing the likelihood that a tissue graft will not be rejected (i.e., decreasing or abrogating the immune response of the recipient to the transplanted tissue) by the subject are well known in the medical arts.

A method is also provided for enhancing survival of retinal cells including retinal neuronal cells, particularly photoreceptor cells and/or ganglion cells and/or amacrine cells, by administering bioactive agents identified according to the methods described herein. These agents may be suitable for treatment of neurological diseases or disorders in general, and for treatment of degenerative diseases of the eye and brain in particular. Neurodegenerative diseases or disorders for which the methods described herein may be useful for treating, curing, impairing preventing, ameliorating the symptoms of, or slowing or stopping the progression of, include but are not limited to glaucoma, macular degeneration, diabetic retinopathy, retinal detachment, retinal blood vessel (artery or vein) occlusion, retinitis pigmentosa, inflammatory retinal disease, optical neuropathy, and retinal disorders associated with other neurodegenerative diseases such as Alzheimer's disease, multiple sclerosis, or Parkinson's Disease, or other conditions such as AIDS.

Bioactive agents that enhance survival of photoreceptor cells may be particularly useful for treating retinal diseases that include photoreceptor neurodegeneration as a sequela of the disease, including but not limited to the dry form of macular degeneration. As described herein, dry or atrophic macular degeneration results in the loss of RPE cells and photoreceptors and is characterized by diminished retinal function due to an overall atrophy of the cells. In contrast, the wet form or neovascular form of macular degeneration involves proliferation of abnormal choroidal vessels, which penetrate the Bruch's membrane and RPE layer into the subretinal space, thereby forming extensive clots and/or scars (see, e.g., Hamdi et al., Front. Biosci. 8:e305-14 (2003)).

Macular degeneration as described herein is a disorder that affects the macula (central region of the retina) and results in the decline and loss of central vision. Age-related macular degeneration occurs typically in individuals over the age of 55 years. The etiology of age-related macular degeneration may include both an environmental influence and a genetic component (see, e.g., Iyengar et al., Am. J. Hum. Genet. 74:20-39 (2004) (Epub 2003 December 19); Kenealy et al., Mol. Vis. 10:57-61 (2004); Gorin et al., Mol. Vis. 5:29 (1999)). More rarely, macular degeneration occurs in younger individuals, including children and infants, and generally the disorder results from a genetic mutation. Types of juvenile macular degeneration include Stargardt's disease (see, e.g., Glazer et al., Ophthalmol. Clin. North Am. 15:93-100, viii (2002); Weng et al., Cell 98:13-23 (1999)); Best's vitelliform macular dystrophy (see, e.g., Kramer et al., Hum. Mutat. 22:418 (2003); Sun et al., Proc. Natl. Acad. Sci. USA 99:4008-13 (2002)), Doyne's honeycomb retinal dystrophy (see, e.g., Kermani et al., Hum. Genet. 104:77-82 (1999)); Sorsby's fundus dystrophy, Malattia Levintinese, fundus flavimaculatus, and autosomal dominant hemorrhagic macular dystrophy (see also Seddon et al., Ophthalmology 108:2060-67 (2001); Yates et al., J. Med. Genet. 37:83-7 (2000); Jaakson et al., Hum. Mutat. 22:395-403 (2003)).

As used herein, a patient (or subject) may be any mammal, including a human, that may have or be afflicted with a neurodegenerative disease or condition or that may be free of detectable disease. Accordingly, the treatment may be administered to a subject who has of an existing disease, or the treatment may be prophylactic, administered to a subject who is at risk for developing the disease or condition. A pharmaceutical composition may be a sterile aqueous or non-aqueous solution, suspension or emulsion, which additionally comprises a physiologically acceptable carrier (pharmaceutically acceptable or suitable carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable carrier known to those of ordinary skill in the art may be employed in the pharmaceutical compositions described herein. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, intraocular, subconjunctival, topical, oral, nasal, intrathecal, rectal, vaginal, sublingual or parenteral administration, including subcutaneous, intravenous, intramuscular, intrastemal, intracavemous, intrameatal or intraurethral injection or infusion. For parenteral administration, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition or a composition that is delivered ocularly is preferably sterile.

Bioactive agents identified according to the methods described herein may be formulated for sustained or slow release. Such compositions may generally be prepared using well known technology and administered by, for example, oral, ocular, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Systemic drug absorption of a drug or composition administered via an ocular route is known to those skilled in the art (see, e.g., Lee et al., Int. J. Pharm. 233:1-18 (2002)). A therapeutic bioactive agent may be delivered by a topical ocular delivery method (see, e.g., Curr. Drug Metab. 4:213-22 (2003)).

Pharmaceutical compositions may be administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the agent(s) in an amount sufficient to provide therapeutic and/or prophylactic benefit (e.g., an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with neurodegeneration of retinal neuronal cells. Optimal doses may generally be determined using experimental models and/or clinical trials. The optimal dose may depend upon the body mass, weight, or blood volume of the patient. The dose depending upon any one of the aforementioned parameters may vary from 1 ng/ml to 10 mg/ml.

The following Examples are offered by way of illustration and not by way of limitation.

### EXAMPLES

### Example 1

### Preparation of Retinal Neuronal Cell Culture System

This Example describes methods for preparing a long-term culture of retinal neuronal cells.

All compounds and reagents were obtained from Sigma Aldrich Chemical Corporation (St. Louis, Mo.) except as noted.

Retinal Neuronal Cell Culture

Porcine eyes were obtained from Kapowsin Meats, Inc. (Graham, Wash.). Eyes were enucleated, and muscle and tissue were cleaned away from the orbit. Eyes were cut in half along their equator and the neural retina was dissected from the anterior part of the eye in buffered saline solution, according to standard methods known in the art. Briefly, the retina, ciliary body, and vitreous were dissected away from the anterior half of the eye in one piece, and the retina was gently detached from the clear vitreous. Each retina was dissociated with papain (Worthington Biochemical Corporation, Lakewood, N.J.), followed by inactivation with fetal bovine serum (FBS) and addition of 134 Kunitz units/ml of DNaseI. The enzymatically dissociated cells were triturated and collected by centrifugation, resuspended in Dulbecco's modified Eagle's medium (DMEM)/F12 medium (Gibco BRL, Invitrogen Life Technologies, Carlsbad, Calif.) containing 25 μg/ml of insulin, 100 μg/ml of transferrin, 60 μM putrescine, 30 nM selenium, 20 nM progesterone, 100 U/ml of penicillin, 100 μg/ml of streptomycin, 0.05 M Hepes, and 10% FBS. Dissociated primary retinal cells were plated onto Poly-D-lysine- and Matrigel- (BD, Franklin Lakes, N.J.) coated glass coverslips that were placed in 24-well tissue culture plates (Falcon Tissue Culture Plates, Fisher Scientific, Pittsburgh, Pa.). Cells were maintained in culture for 5 days to one month in 0.5 ml of media (as above, except with only 1% FBS) at 37° C. and 5% CO₂.

Immunocytochemistry Analysis

The retinal neuronal cells were cultured for 1, 3, 6, and 8 weeks, and the cells were analyzed by immunohistochemistry at each time point. Immunocytochemistry analysis was performed according to standard techniques known in the art. Rod photoreceptors were identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) was used to identify ganglion cells; an antibody to β3-tubulin (G7121 mouse monoclonal, diluted 1:1000, Promega, Madison, Wis.) was used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) were used to identify subpopulations of calbindin- and calretinin-expressing interneurons in the inner nuclear layer. Briefly, the retinal cell cultures were fixed with 4% paraformaldehyde (Polysciences, Inc, Warrington, Pa.) and/or ethanol, rinsed in Dulbecco's phosphate buffered saline (DPBS), and incubated with primary antibody for 1 hour at 37° C. The cells were then rinsed with DPBS, incubated with a secondary antibody (Alexa 488- or Alexa 568-conjugated secondary antibodies (Molecular Probes, Eugene, Oreg.)), and rinsed with DPBS. Nuclei were stained with 4′,6-diamidino-2-phenylindole (DAPI, Molecular Probes), and the cultures were rinsed with DPBS before removing the glass coverslips and mounting them with Fluoromount-G (Southern Biotech, Birmingham, Ala.) on glass slides for viewing and analysis.

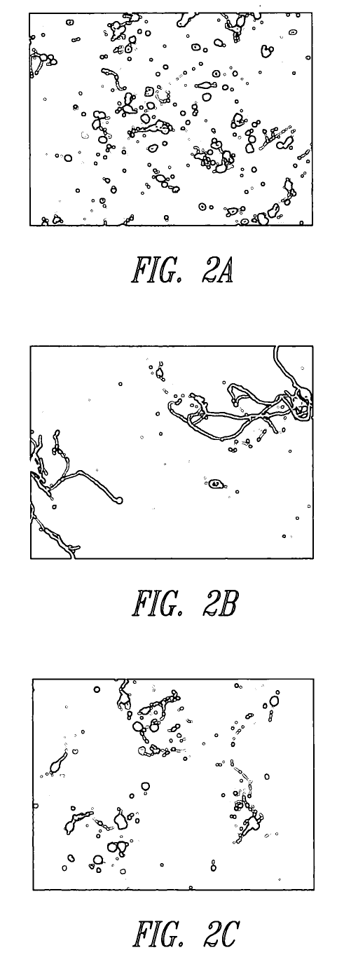

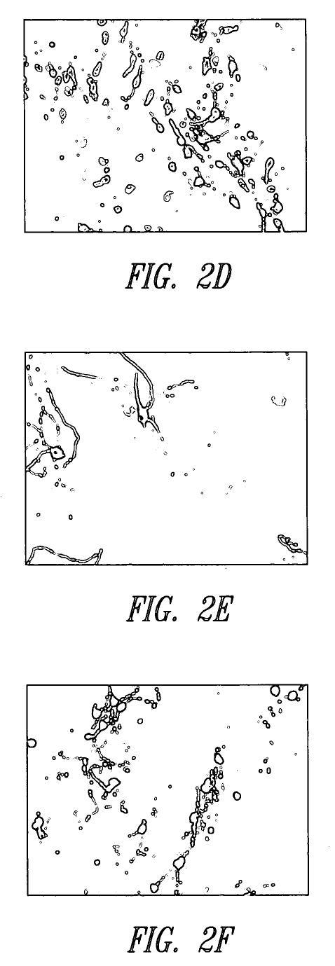

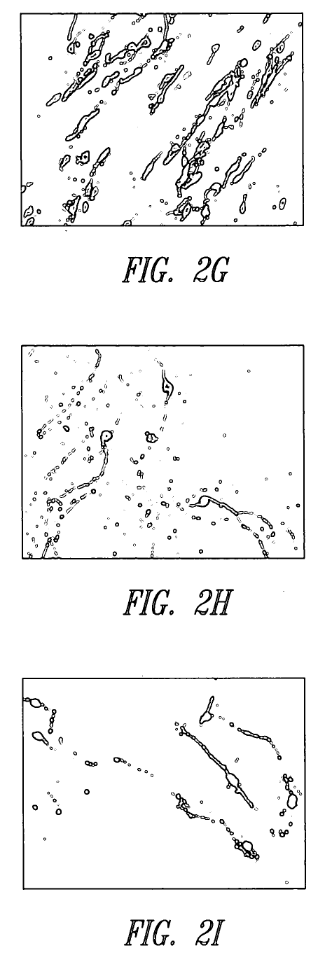

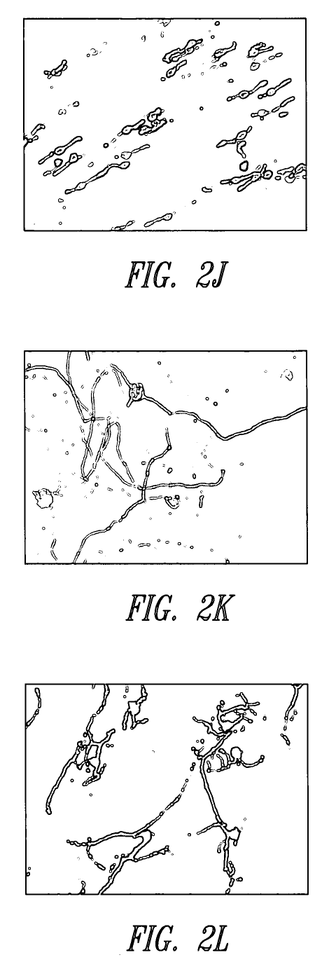

FIG. 2 illustrates survival of primate mature retinal neurons after varying times in culture. Porcine retinal cells were cultured for 1 week (FIG. 2A, 2B, 2C); 3 weeks (FIG. 2D, 2E, 2F); 6 weeks (FIG. 2G, 2H, 2K); and 8 weeks (FIG. 2J, 2K, 2L). Photoreceptor cells were identified using a rhodopsin antibody (FIG. 2A, 2D, 2G, 2J); ganglion cells were identified using an NFM antibody (FIG. 2B, 2E, 2H, 2K); and amacrine and horizontal cells were identified by staining with an antibody specific for calretinin (FIG. 2C, 2F, 2I, 2L).

### Example 2

### White Light Induced Stress of Retinal Neuronal Cells

This Example describes the effects of white light-induced stress on retinal neuronal cells neuronal cells cultured in an extended retinal cell culture system.

White Light-Induced Stress

A device was built to uniformly deliver light of specified wavelengths to specified wells of the 24-well plates. The device contained a fluorescent cool white bulb (GE P/N FC12T9/CW) wired to an AC power supply. The bulb was mounted inside a standard tissue culture incubator. White light stress was applied by placing plates of cells directly underneath the fluorescent bulb. The CO₂ levels were maintained at 5%, and the temperature at the cell plate was maintained at 37° C. The temperature was monitored by using thin thermocoupies.

The light intensities for all devices were measured and adjusted using a light meter from Extech Instruments Corporation (P/N 401025; Waltham, Mass.). Mature retinal cell cultures were prepared as described in Example 1. Cultures were subjected to light stress for 0, 2, 4, 8, 24, and 48 hours at intensities including 1000, 1200, 2000, 2500, 4000, and 6000 lux (as measured by the same Extech light meter). Following exposure to white light, the cells rested for 14-16 hours. The cells were then analyzed by immunocytochemistry.

Immunocytochemistry Analysis of Retinal Cell Cultures

Immunocytochemical analysis was performed according to standard techniques known in the art. Rod photoreceptors were identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) was used to identify ganglion cells; an antibody to β3-tubulin (G7121 mouse monoclonal, diluted 1:1000, Promega, Madison, Wis.) was used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) were used to identify subpopulations of calbindin- and calretinin-expressing interneurons in the inner nuclear layer.

Briefly, the retinal cell cultures were fixed with 4% paraformaldehyde (Polysciences, Inc, Warrington, Pa.) and/or ice-cold methanol, rinsed in Dulbecco's phosphate buffered saline (DPBS), and incubated with primary antibody for 1 hour at 37° C. or overnight at 4° C. The cells were then rinsed with DPBS, incubated with a secondary antibody (Alexa 488- or Alexa 568-conjugated secondary antibodies (Molecular Probes, Eugene, Oreg.)), and rinsed with DPBS. Nuclei were stained with 4′,6-diamidino-2-phenylindole (DAPI, Molecular Probes), and the cultures were rinsed with DPBS before removing the glass coverslips and mounting them with Fluoromount-G (Southern Biotech, Birmingham, Ala.) on glass slides for viewing and analysis.

Cultures were analyzed by counting rhodopsin-labeled photoreceptors and NFM-labeled ganglion cells using an Olympus IX81 or CZX41 microscope (Olympus, Tokyo, Japan). Twenty fields of view were counted per coverslip with a 20× objective lens. Six coverslips were analyzed by this method for each condition in each experiment. Cells that were not exposed to any stressor were counted, and cells exposed to a stressor were normalized to the number of cells in the control.

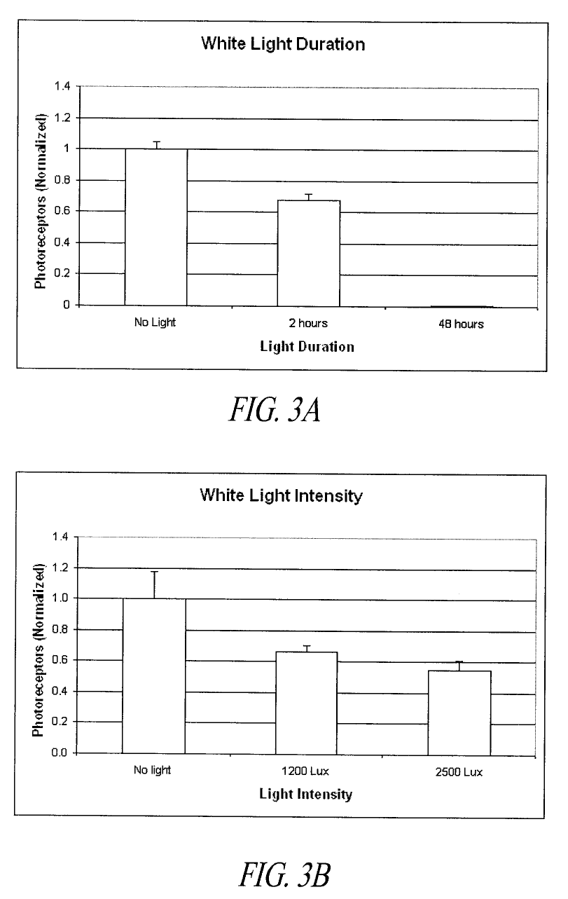

Representative data are presented in FIGS. 3 and 4. Data were analyzed using the unpaired Student's t-test. FIG. 3 shows the effect on photoreceptor cells when exposed to varying duration of white light (FIG. 3A) and on photoreceptor cells exposed to varying light intensity (FIG. 3B). Photoreceptor cells showed a dose response to both duration and intensity of white light, and NFM-expressing ganglion cells did not show a response to 6000 lux of white light stress for 24 hours (FIG. 4). The number of photoreceptor cells detected using the rhodopsin-specific antibody in the presence of light stress was statistically different from the number of cells detected in the absence of white light stress at a greater than 95% confidence level. The number of ganglion cells detected using the NFM specific antibody in the absence of light stress was not statistically different from the number of cells detected in the presence of white light stress at a greater than 95% confidence level.

Apoptosis Analysis

Retinal cell cultures were cultured for 2 weeks and then exposed to white light stress at 6000 lux for 24 hours followed by a 13-hour rest period. To assess apoptosis, TUNEL was performed according to standard techniques known in the art and according to the manufacturer's instructions. Briefly, the retinal cell cultures were first fixed with 4% paraformaldehyde and then ethanol, rinsed in DPBS. The fixed cells were then incubated with TdT enzyme (0.2 units/μl final concentration) in reaction buffer (Fermentas, Hanover, Md.) combined with Chroma-Tide Alexa 568-5-dUTP (0.1 μM final concentration) (Molecular Probes) for 1 hour at 37° C. Cultures were rinsed with DPBS, and incubated with primary antibody either overnight at 4° C. or for 1 hour at 37° C. The cells were then rinsed with DPBS, incubated with Alexa 488-conjugated secondary antibodies, and rinsed with DPBS. Nuclei were stained with DAPI, and the cultures were rinsed with DPBS before removing the glass coverslips and mounting them with Fluoromount-G on glass slides for viewing and analysis.

Cultures were analyzed by counting TUNEL-labeled nuclei using an Olympus IX81 or CZX41 microscope (Olympus, Tokyo, Japan). Twenty fields of view were counted per coverslip with a 20× objective lens. Six coverslips were analyzed by this method for each condition. Cells that were not exposed to any stressor were counted, and cells exposed to a stressor were normalized to the number of cells in the control.

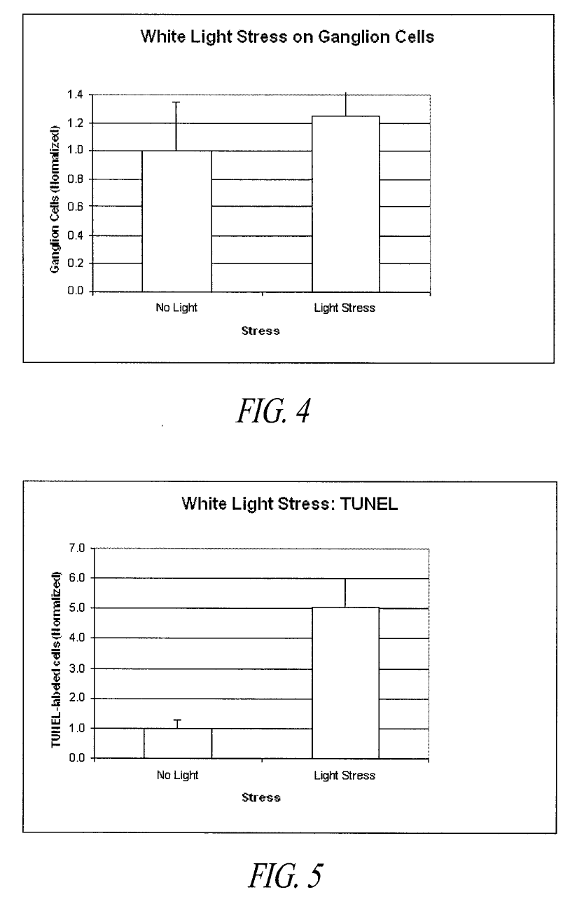

FIG. 5 shows that TUNEL-labeling increased 5-fold after 6000 lux of white light stress for 24 hours. Data were analyzed using the unpaired Student's t-test. The number of TUNEL-labeled retinal cells exposed to white light stress was statistically different at a greater than 95% confidence level from the number of TUNEL-labeled retinal cells that were not exposed to the light stress.

### Example 3

### Blue Light Induced Stress of Retinal Neuronal Cells

This Example describes the effects of blue light-induced stress on retinal neuronal cells cultured in an extended retinal cell culture system

Blue Light-Induced Stress

Retinal cell cultures were prepared as described in Example 1. After culturing the cells for 1 week, a blue light stress was applied. Blue light was delivered by a custom-built light-source, which consisted of two arrays of 24 (4×6) blue light-emitting diodes (Sunbrite LED P/N SSP-01TWB7UWB12), designed such that each LED was registered to a single well of a 24 well disposable plate. The first array was placed on top of a 24 well plate full of cells, while the second one was placed underneath the plate of cells, allowing both arrays to provide a light stress to the plate of cells simultaneously. The entire apparatus was placed inside a standard tissue culture incubator. The CO₂ levels were maintained at 5%, and the temperature at the cell plate was maintained at 37° C. The temperature was monitored by using thin thermocouples. Current to each LED was controlled individually by a separate potentiometer, allowing a uniform light output for all LEDs. Cell plates were exposed to 2000 lux of blue light stress for either 2 hours or 48 hours, followed by a 14 hour rest period.

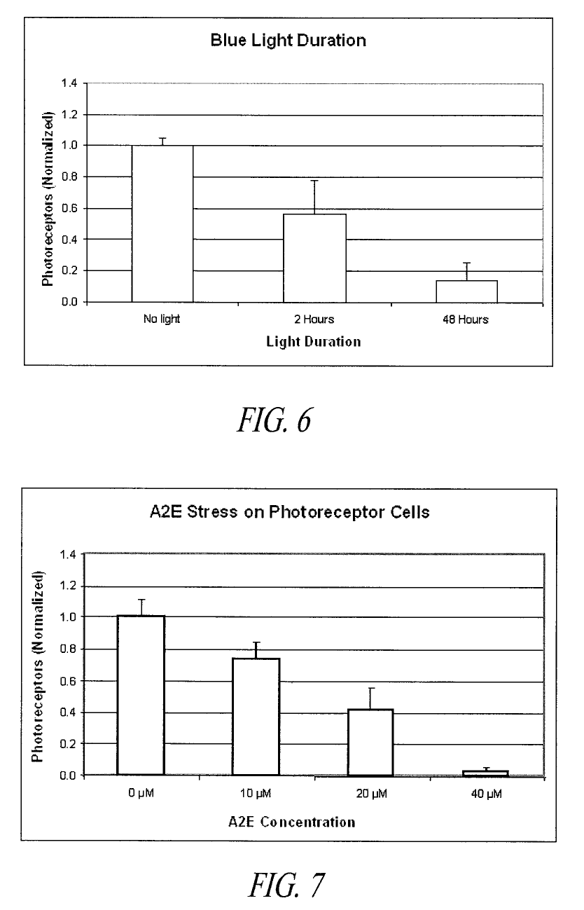

Immunochemistry analysis was performed and the data analyzed as described in Examples 1 and 2. The number of rhodopsin-expressing photoreceptors decreased after 2000 lux of blue light stress, demonstrating a dose response to stress duration (FIG. 6). The data were statistically different at a greater than 95% confidence level (unpaired Student's t-test).

### Example 4

### A2E-Induced Stress of Retinal Neuronal Cells

This Example for the effects of A2E-induced stress of retinal neuronal cells cultured in an extended retinal cell culture system.

A2E-Induced Stress

Retinal cell cultures were prepared as described in Example 1. After culturing the cells for 1 week, a chemical stress, A2E, was applied. A2E was obtained from Dr. Koji Nakanishi (Columbia University, New York City, N.Y.). A2E was diluted in ethanol and added to the retinal cell cultures at concentration of 0, 10 μM, 20 μM, and 40 μM. Cultures were treated for 24 and 48 hours. The cultures were maintained in tissue culture incubators for the duration of the stress at 37° C. and 5% CO₂.

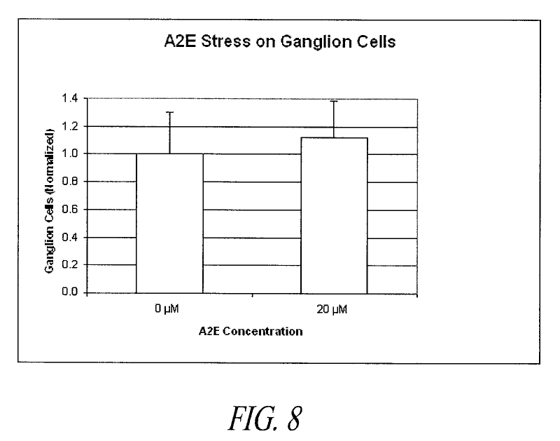

Immunocytochemical analysis was performed and the data analyzed as described in Examples 1 and 2. The number of rhodopsin-expressing photoreceptors showed a dose response to varying concentrations of A2E after 24 hours (FIG. 7 (statistical difference at a greater than 95% confidence level; unpaired Student's t-test), whereas the number of NFM-expressing ganglion cells was not statistically different after 24 hours of no stress or exposure to 20 μM A2E stress (FIG. 8) (unpaired Student's t-test; greater than 95% confidence level).

### Example 5

### Effect of White LED Light-Induced Stress on Retinal Neuronal Cells

This Example describes the effect of white LED light-induced stress of retinal cells cultured in an extended retinal cell culture system.

White LED Light-Induced Stress

Retinal cell cultures are prepared as described in Example 1. White light is delivered by a custom built LED light-source, which consists of two arrays of 24 (4×6) white light-emitting diodes (Sunbrite LED P/N SSP-01TWB9WB12), designed as in Example 3. Retinal cells are analyzed by immunocytochemistry procedures and the data are analyzed according to methods described in Examples 1 and 2. White LED light-induced stress causes intensity and duration-dependent decreases in the number of photoreceptors, whereas ganglion cell numbers remain constant.

### Example 6

### Cigarette Smoke Condensate Induced Stress of Retinal Neuronal Cells

This Example describes the effects of cigarette smoke condensate stress on retinal neuronal cells cultured in an extended retinal cell culture system.

Cigarette Smoke Condensate Induced Stress

Retinal cell cultures were prepared as described in Example 1. Cells were maintained in culture for 5 days to one month in 0.5 ml of media (as above, except with only 1% FBS) at 37° C. and 5% CO₂. Cigarette Smoke Condensate (CSC) was obtained from Murty Pharmaceuticals (Lexington, Ky.). Briefly, CSC was prepared at the University of Kentucky by smoking 1R3F Standard Research Cigarettes on an FTC Smoke Machine. CSC was collected on a filter, and the Total Particulate Matter (TPM) on the filter was calculated by the weight gain of the filter. From the TPM, the amount of DMSO to prepare a theoretical 4% (w/v) solution used for extraction was then calculated. The condensate was extracted with DMSO by soaking the filter in DMSO and sonicating the filter. The extracted CSC was then packaged in 1 mL vials and stored at −70° C.

The retinal neuronal cell culture prepared as described above was exposed to 100 μg/mL CSC for 24 hours under normal tissue culture conditions of 37° C. and 5% CO₂.

Immunocytochemistry Analysis of Retinal Cell Cultures

Immunocytochemical analysis was performed as described in Examples 1 and 2 according to standard techniques known in the art. Rod photoreceptors were identified by labeling with a rhodopsin-specific antibody (mouse monoclonal, diluted 1:500; Chemicon International, Temecula, Calif.). An antibody to mid-weight neurofilament (NFM rabbit polyclonal, diluted 1:10,000, Chemicon) was used to identify ganglion cells; an antibody to β3-tubulin (G7121 mouse monoclonal, diluted 1:1000, Promega, Madison, Wis.) was used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) were used to identify subpopulations of calbindin- and calretinin-expressing interneurons in the inner nuclear layer.

Briefly, the retinal cell cultures were fixed with 4% paraformaldehyde (Polysciences, Inc, Warrington, Pa.) and/or ice-cold methanol, rinsed in Dulbecco's phosphate buffered saline (DPBS), and incubated with primary antibody for 1 hour at 37° C. or overnight at 4° C. The cells were then rinsed with DPBS, incubated with a secondary antibody (Alexa 488- or Alexa 568-conjugated secondary antibodies (Molecular Probes, Eugene, Oreg.)), and rinsed with DPBS. Nuclei were stained with 4′,6-diamidino-2-phenylindole (DAPI, Molecular Probes), and the cultures were rinsed with DPBS before removing the glass coverslips and mounting them with Fluoromount-G (Southern Biotech, Birmingham, Ala.) on glass slides for viewing and analysis.

Cultures were analyzed by counting rhodopsin-labeled photoreceptors and NFM-labeled ganglion cells using an Olympus IX81 or CZX41 microscope (Olympus, Tokyo, Japan). Twenty fields of view were counted per coverslip with a 20× objective lens. Six coverslips were analyzed by this method for each condition in each experiment. Cells that were not exposed to any stressor were counted, and cells exposed to a stressor were normalized to the number of cells in the control.

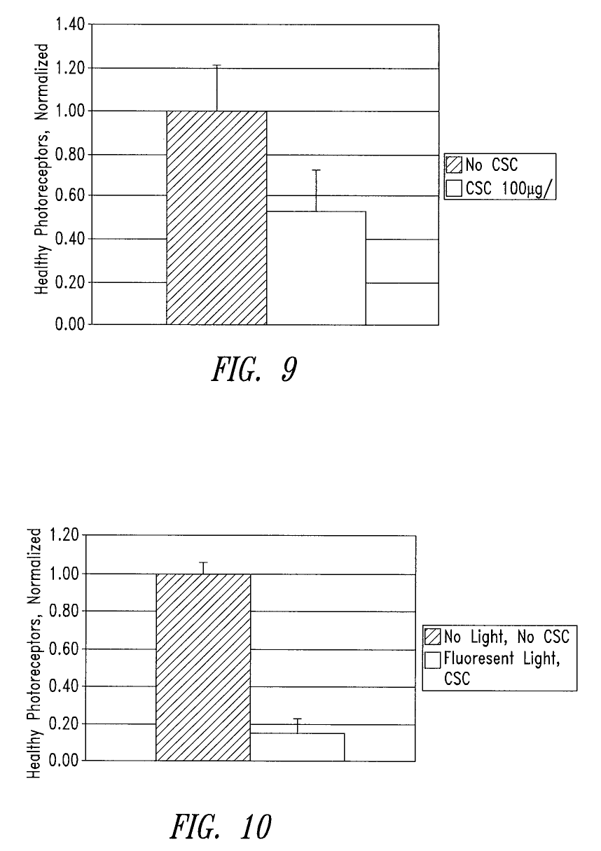

Representative normalized data are presented in FIG. 9. Data were analyzed using the unpaired Student's t-test. FIG. 9 shows the effect on photoreceptor cells when the cells were exposed to cigarette smoke condensate stress. The number of photoreceptor cells detected using the rhodopsin-specific antibody in the presence of cigarette smoke condensate stress was statistically smaller than the number of cells detected in the absence of stress at a greater than 95% confidence level.

### Example 7

### Cigarette Smoke Condensate Plus Light Induced Stress of Retinal Neuronal Cells

This Example describes the effects of cigarette smoke condensate plus light-induced stress on retinal neuronal cells cultured in an extended retinal cell culture system. Retinal cell cultures were prepared as described in Example 1.

Cigarette Smoke Condensate Plus Light Induced Stress

The device described in Example 2 to uniformly deliver light of specified wavelengths to specified wells of 24-well tissue culture plates. The fluorescent cool white bulb of the device was mounted inside a standard tissue culture incubator. White light stress was applied by placing plates of cells directly underneath the fluorescent bulb. The CO₂ level in the tissue culture incubator was maintained at 5%, and the temperature at the cell plate was maintained at 37° C. The temperature was monitored by using thermocouples.

The light intensities for all devices were measured and adjusted using a light meter from Extech Instruments Corporation (P/N 401025; Waltham, Mass.). After the cells were cultured for one week, the cell cultures were subjected to light stress for 24 hours at an intensity of 1500 lux. The cells were then analyzed by immunocytochemistry.

To another sample of cells cultured for 1 week in the absence of any stress, cigarette smoke condensate (100 μg/ml) was added to the cultures and light stress was applied. The cultures were maintained for 24 hours in the presence of both stressors. The CO₂ levels were maintained at 5%, and the temperature at the cell plate was maintained at 37° C.

Immunochemistry analysis was performed and the data were analyzed as described in Examples 1 and 2. FIG. 10 presents representative data. The number of rhodopsin-expressing photoreceptors decreased after light plus cigarette smoke condensate stress. The data were statistically different at a greater than 95% confidence level for cells exposed to the stressors compared to cells not exposed to the cell stressors (unpaired Student's t-test).

### Example 8

### Pressure Induced Stress of Retinal Neuronal Cells

This Example describes the effects of elevated atmospheric pressure on retinal neuronal cells cultured in an extended retinal cell culture system. Retinal cell cultures were prepared as described in Example 1, and just before undergoing pressure stress, the tissue culture media was changed from media with serum to media without serum.

A pressure chamber was built to subject cells to a positive gage pressure of 75 mmHg. This chamber was placed inside a standard tissue culture incubator and was pressurized using a canister of gas containing 5% CO₂ and 95% air to match the conditions within the incubator. The CO₂ levels were maintained at 5%, and the temperature at the cell plate was maintained at 37° C. The temperature was monitored with thermometers.

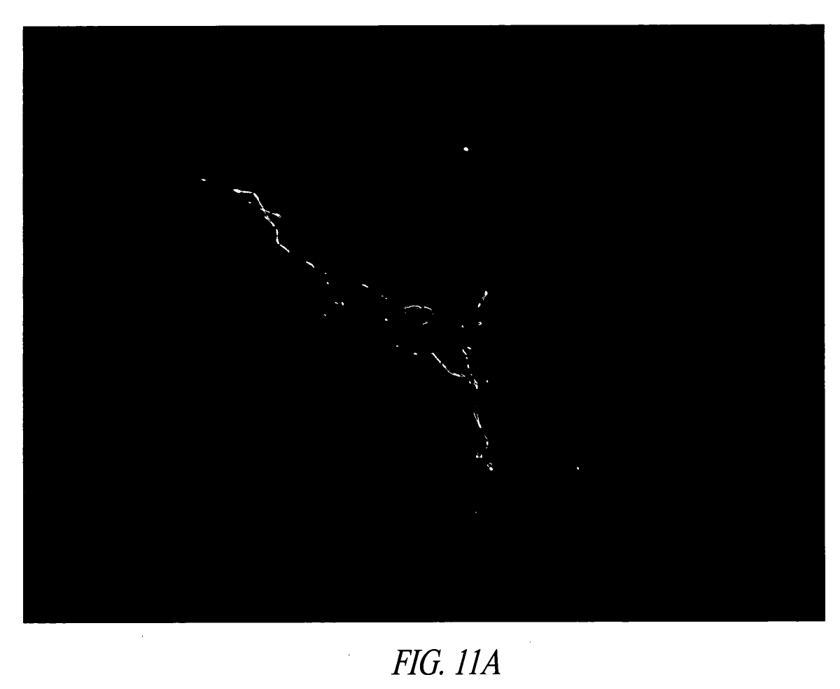

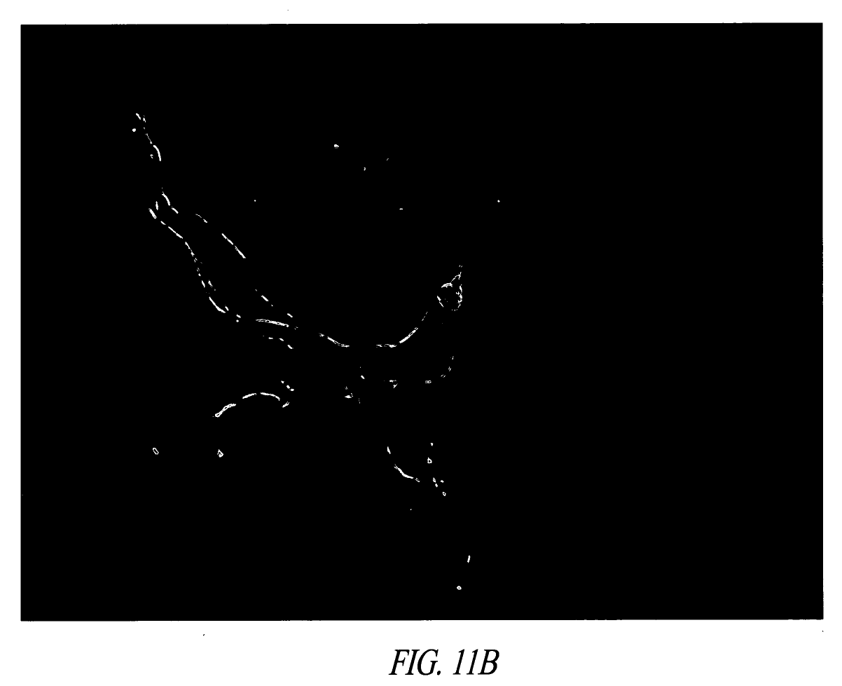

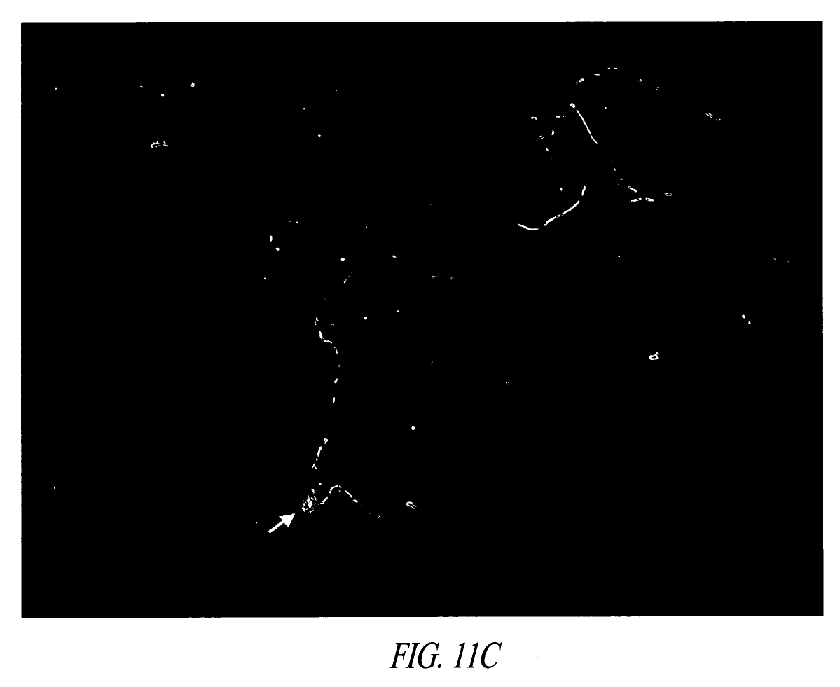

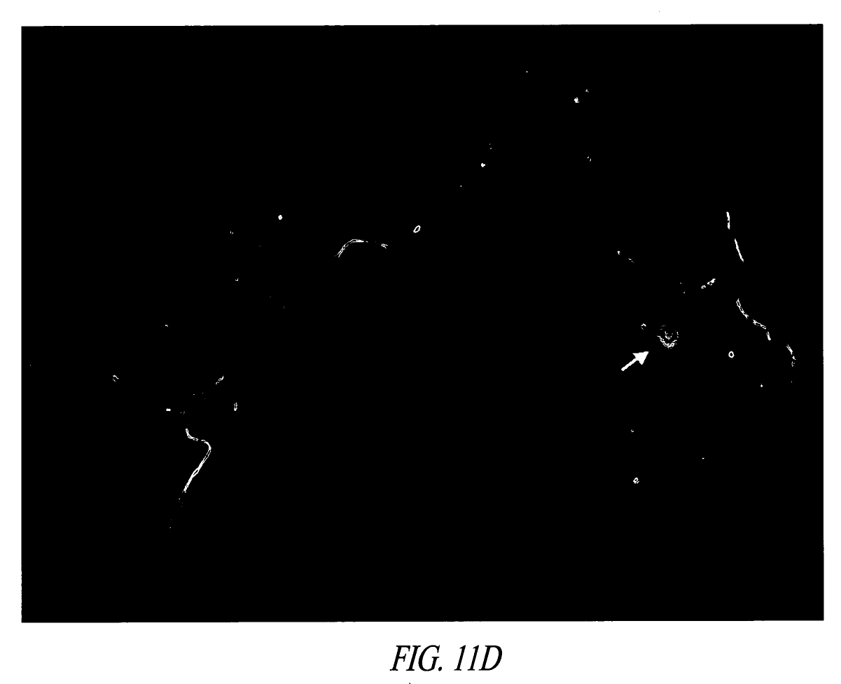

After a 24-hour exposure to elevated pressure, the retinal neuronal cells were analyzed by immunochemistry according to the methods described in Examples 1 and 2. Ganglion cells were detected using a chicken anti-NFM antibody diluted 1:5000 (Chemicon International). The neuronal cells in the culture were also detected using an antibody that specifically binds to the apoptotic marker caspase-3 (rabbit anti-caspase-3, active, diluted 1:5000; R & D Systems, Inc., Minneapolis, Minn.). Binding of the anti-NFM antibody was detected using Alexafluor 594 goat anti-chicken IgG (1:1500) (Molecular Probes), and binding of anti-caspase-3 antibody was detected using Alexafluor 488 goat anti-rabbit IgG (1:1500) (Molecular Probes). FIG. 11A and FIG. 11B show ganglion cells that were not exposed to increased atmospheric pressure as a stressor. FIGS. 11C and 11D illustrate examples of ganglion cells undergoing apoptosis. Ganglion cells detected with anti-caspase-3 antibody are indicated by the arrows.

### Example 9

### EPO Enhances Photoreceptor Survival

This Example describes the use of the mature retinal cell culture system that comprises a cell stressor for determining the effects of an agent on the viability of the retinal cells. Acute hypoxia in the adult mouse retina stimulates expression of erythropoietin (EPO) (Grimm et al., Nat. Med. 8:718-724 (2002)). Accordingly, the effects of EPO on retinal cells were examined.

All compounds and reagents were obtained from Sigma Aldrich Chemical Corporation (St. Louis, Mo.), except as noted.

Retinal cell cultures were prepared as described in Example 1. Erythropoietin (EPO) (R&D Systems, Minneapolis, Minn.) was diluted in phosphate buffered saline (PBS) and added to the culture wells at a final concentration of 1 U/ml for 24 hours at 37° C. and 5% CO₂. The cells were stressed by changing to media that contained both 25 μM A2E (obtained from Dr. Koji Nakanishi, Columbia University, New York City, N.Y.; diluted in ethanol) and 1 U/ml EPO and incubated for 24 hours.

Immunohistochemistry Analysis

Immunohistochemistry analysis was performed as described in Examples 1 and 2 according to standard methods used in the art. Rod photoreceptors were identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) was used to identify ganglion cells; an antibody to beta3-tubulin was used to generally identify interneurons, and antibodies to calbindin and calretinin were used to identify subpopulations of calbindin- and calretinin-expressing interneurons in the inner nuclear layer. Briefly, the retinal cell cultures were fixed with 4% paraformaldehyde (Polysciences, Inc, Warrington, Pa.) and/or ethanol, rinsed in Dulbecco's phosphate buffered saline (DPBS), and incubated with primary antibody for 1 hour at 37° C. The cells were then rinsed with DPBS, incubated with a secondary antibody (Alexa 488- or Alexa 568-conjugated secondary antibodies (Molecular Probes, Eugene, Oreg.)), and rinsed with DPBS. Nuclei were stained with 4′,6-diamidino-2-phenylindole (DAPI, Molecular Probes), and the cultures were rinsed with DPBS before removing the glass coverslips and mounting them with Fluoromount-G (Southern Biotech, Birmingham, Ala.) on glass slides for viewing and analysis.

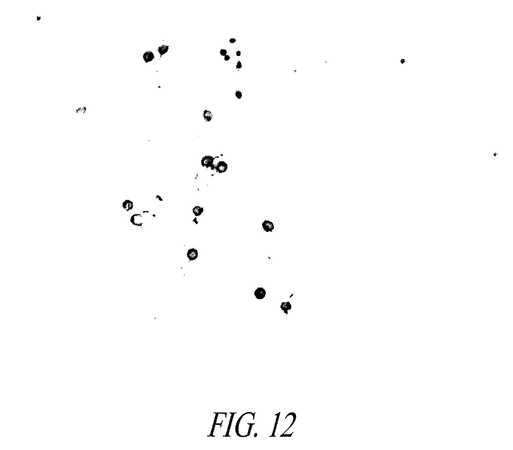

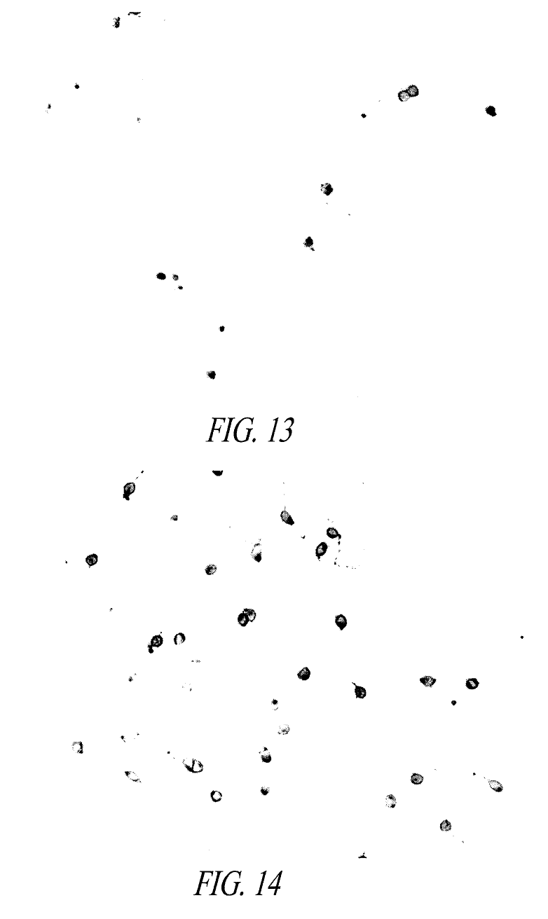

Cultures were analyzed by counting rhodopsin-labeled photoreceptors and NFM-labeled ganglion cells using an Olympus IX81 or CZX41 microscope (Olympus, Tokyo, Japan). Twenty fields of view were counted per coverslip with a 20× objective lens. Six coverslips were analyzed by this method for each condition in each experiment. Cells that were not exposed to either EPO or to any stressor were counted, and cells exposed to a stressor with or without treatment with EPO were normalized to the number of cells in the control. FIG. 12 shows representative rhodopsin-expressing photoreceptors before stress. FIG. 13 shows representative rhodopsin-expressing photoreceptors after stress (A2E, 25 μM for 24 hours). The small dots indicate debris; the total live cell count is much smaller than in FIG. 1. FIG. 14 shows rhodopsin-expressing photoreceptors under stress but with addition of EPO (1 U/mL) for the same duration. The live cell count is much greater than it is in FIG. 13, indicating neuroprotection of photoreceptors.

From the foregoing, it will be appreciated that, although specific embodiments of the invention have been described herein for purposes of illustration, various modifications may be made without deviating from the spirit and scope of the invention.

1. A cell culture system comprising a plurality of mature retinal cells and at least one cell stressor, wherein the cell stressor reduces viability of the mature retinal cells, wherein the plurality of mature retinal cells comprises a plurality of retinal neuronal cells, retinal pigmented epithelial cells, and Müller glial cells, and wherein the cell culture system is substantially free of cells from non-retinal tissue.

2. The cell culture system of claim 1 wherein the cell stressor is light, retinoid N-retinylidene-N-retinyl-ethanolamine (A2E) or an isoform thereof, or cigarette smoke condensate.

3. The cell culture system of claim 1 wherein the cell stressor is light, and wherein the light is blue light or white light.

4. (canceled)

5. The cell culture system of claim 3 wherein the light is blue light and wherein the blue light has an intensity between about 250-8000 lux.

6. (canceled)

7. The cell culture system of claim 3 wherein the light is white light and wherein the white light has an intensity between about 250-8000 lux.

8.-9. (canceled)

10. The cell culture system of claim 1 wherein the cell stressor is retinoid N-retinylidene-N-retinyl-ethanolamine (A2E) or an isoform thereof.

11. The cell culture system of claim 1 wherein the cell stressor is cigarette smoke condensate.

12. (canceled)

13. The cell culture system of claim 1 comprising two or more cell stressors selected from light, A2E, and cigarette smoke condensate.

14. The cell culture system of claim 13 wherein at least two cell stressors are light and A2E.

15. The cell culture system of claim 13 wherein at least two cell stressors are light and cigarette smoke condensate.

16. (canceled)

17. The cell culture system according to claim 1 wherein the plurality of retinal neuronal cells comprises at least one bipolar cell, at least one horizontal cell, at least one amacrine cell, at least one ganglion cell, and at least one photoreceptor cell.

18.-27. (canceled)

28. A cell culture system comprising a plurality of mature retinal cells, wherein the cell culture system is substantially free of cells from a non-retinal tissue.

29. The cell culture system according to claim 28 wherein the plurality of mature retinal cells comprises a plurality of retinal neuronal cells, retinal pigmented epithelial cells, and Müller glial cells.

30.-38. (canceled)

39. A method for producing the cell culture system of claim 1 comprising: (a) isolating the plurality of mature retinal cells from a biological source; (b) culturing the mature retinal cells under conditions that maintain viability of the mature retinal cells; and (c) adding the at least one cell stressor.

40. The method of claim 39 wherein the biological source is retinal tissue from a mammal or a bird.

41. The method of claim 40 wherein the mammal is a human, a pig, a non-human primate, an ungulate, a dog, or a rodent.

42.-43. (canceled)

44. A method for identifying a stressor of mature retinal cells comprising: (a) contacting a candidate stressor and a cell culture system wherein the cell culture system comprises a plurality of mature retinal cells, said plurality of mature retinal cells comprising a plurality of retinal neuronal cells, retinal pigmented epithelial cells, and Müller glial cells, and wherein the cell culture system is substantially free of cells from non-retinal tissue, under conditions and for a time sufficient to permit interaction between the candidate stressor and the mature retinal cells; and (b) comparing viability of a plurality of mature retinal cells in the presence of the candidate stressor with viability of a plurality of mature retinal cells in the absence of the candidate stressor, and therefrom identifying a stressor of retinal cells.

45. The method of claim 44 wherein viability is determined by comparing a level of survival of the plurality of mature retinal cells in the presence of the candidate stressor with a level of survival of the plurality of mature retinal cells in the absence of the candidate stressor, wherein decreased survival in the presence of the candidate agent indicates that the stressor decreases viability of the retinal cells.

46. The method of claim 44 wherein viability is determined by comparing degeneration of the plurality of mature retinal cells in the presence of the candidate stressor with degeneration of the plurality of mature retinal cells in the absence of the candidate stressor, wherein enhancement of degeneration in the presence of the candidate stressor indicates that the stressor decreases viability of the retinal cell.

47. The method of claim 44 wherein the step of comparing viability of the plurality of mature retinal cells comprises determining viability of at least one retinal cell type that is selected from a retinal neuronal cell, a retinal pigmented epithelial cell, and a Müller glial cell.

48. The method of claim 44-47 wherein the at least one retinal neuronal cell is an amacrine cell, a horizontal cell, a ganglion cell, a bipolar cell, or a photoreceptor cell.

49.-52. (canceled)

53. A method for identifying a bioactive agent that alters viability of a mature retinal cell comprising: (a) contacting a candidate agent and the cell culture system of claim 1, under conditions and for a time sufficient to permit interaction between a mature retinal cell of the cell culture system and the candidate agent; and (b) comparing viability of a mature retinal cell in the presence of the candidate agent with viability of a mature neuronal cell in the absence of the candidate agent, therefrom identifying a bioactive agent that is capable of altering viability of a retinal cell.

54. The method of claim 53 wherein viability is determined by comparing a level of survival the mature retinal cell in the presence of the candidate agent with a level of survival of the mature retinal cell in the absence of the candidate agent, wherein increased survival in the presence of the candidate agent indicates that the agent increases viability of the retinal cell.

55. The method of claim 53 wherein viability is determined by comparing neurodegeneration of the mature retinal cell in the presence of the candidate agent with neurodegeneration of the mature retinal cell in the absence of the candidate agent, wherein inhibition of neurodegeneration in the presence of the candidate agent indicates that the agent increases viability of the retinal cell.

56. The method of claim 53 wherein the step of comparing viability of the mature retinal cell comprises determining viability of (a) at least one retinal neuronal cell selected from a bipolar cell, a horizontal cell, an amacrine cell, a ganglion cell, and a photoreceptor cell; (b) at least one retinal pigmented epithelial cell; or (c) at least one Müller glial cell.

57. A method for identifying a bioactive agent capable of treating a retinal disease comprising: (a) contacting a candidate agent with a cell culture system according to claim 1, under conditions and for a time sufficient to permit interaction between a mature retinal cell of the cell culture system and the candidate agent; and (b) comparing viability of a mature retinal cell in the cell culture system in the presence of the candidate agent with viability of a mature retinal cell in the absence of the candidate agent, wherein an increase in viability of the mature retinal cell in the presence of the candidate agent identifies a bioactive agent that is capable of treating a retinal disease.

58. The method according to claim 57 wherein viability is determined by comparing a level of survival of the mature retinal cell in the presence of the candidate agent with a level of survival of the mature retinal cell in the absence of the candidate agent, wherein increased survival in the presence of the candidate agent indicates that the agent increases viability of the retinal cell.

59. The method according to claim 57 wherein viability is determined by comparing degeneration of the mature retinal cell in the presence of the candidate agent with degeneration of the mature retinal cell in the absence of the candidate agent, wherein inhibition of degeneration in the presence of the candidate agent indicates that the agent increases viability of the retinal cell.

60. The method of claim 57 wherein the step of comparing viability of the mature retinal cell comprises determining viability of (a) at least one retinal neuronal cell type that is selected from a bipolar cell, a horizontal cell, an amacrine cell, a ganglion cell, and a photoreceptor cell; (b) at least one retinal pigmented epithelial cell; or (c) at least one Müller glial cell.

61. The method of claim 57 wherein the retinal disease is macular degeneration, glaucoma, diabetic retinopathy, retinal detachment, retinal blood vessel occlusion, retinitis pigmentosa, optic neuropathy, inflammatory retinal disease, or a retinal disorder associated with Alzheimer's disease, Parkinson's disease, or multiple sclerosis.
